(19) United States
(12) Patent Application Publication
(10) Pub. No.: US 2014/0221461 A1
(43) Pub. Date: Aug. 7, 2014

(54) NUCLEIC ACID MOLECULES AND USES THEREOF

(71) Applicant: Atossa Genetics, Inc., Seattle, WA (US)

(72) Inventor: Steven C. QUAY, Seattle, WA (US)

(73) Assignee: Atossa Genetics, Inc., Seattle, WA (US)

(21) Appl. No.: 14/251,255

(22) Filed: Apr. 11, 2014

Related U.S. Application Data
(63) Continuation of application No. 13/392,042, filed on Feb. 23, 2012, now abandoned.
(60) Provisional application No. 61/237,573, filed on Aug. 27, 2009.

(51) Int. Cl. A61K 31/713

(52) U.S. Cl. CPC A61K 31/713
USPC 514/44 A

(57) ABSTRACT

Provided in this application are formulations of double stranded RNA molecules and Krebs Cycle analogs that improving ribonuclease stability, reducing off-target effects of a double stranded siRNA molecule, or of reducing interferon responsiveness of a double stranded siRNA molecule using such dsRNA. Also disclosed are methods of treating a primary tumor or a metastasis by contacting circulating tumor cells, a primary tumor, or a metastasis with a described formulation.

### CROSS-REFERENCE

This application is a continuation of U.S. application Ser. No. 13/392,042, filed Feb. 23, 2012, which is the National Stage entry of International Application No. PCT/US2010/047026, filed Aug. 27, 2010, which claims priority to U.S. Provisional Application No. 61/237,573, filed Aug. 27, 2009, all of which are herein incorporated by reference in their entireties.

### BACKGROUND OF THE INVENTION

RNA interference (also, RNAi) refers to the process of sequence-specific post-transcriptional gene silencing in animals mediated by short interfering RNAs (siRNAs) or micro RNAs (miRNAs).

The presence of long dsRNAs in cells stimulates the activity of a ribonuclease III enzyme referred to as dicer. Dicer is involved in the processing of the dsRNA into short pieces of dsRNA known as short interfering RNAs (siRNAs). Short interfering RNAs derived from dicer activity are typically about 21 to about 23 nucleotides in length and comprise about 19 base pair duplexes. The RNAi response also features an endonuclease complex, commonly referred to as an RNA-induced silencing complex (RISC), which mediates cleavage of single-stranded RNA having sequence complementary to the antisense strand of the siRNA duplex. Cleavage of the target RNA takes place in the middle of the region complementary to the antisense strand of the siRNA duplex.

### SUMMARY OF THE INVENTION

Disclosed herein, in certain embodiments, is a formulation, comprising: (a) an RNA molecule comprising at least one of a locked nucleic acid (LNA), an unlocked nucleic acid (UNA), a bridged nucleic acid (BNA), a glycerol nucleic acid (GNA), or a combination thereof; and (b) an RNAi carrier. In some embodiments, the RNA molecule is an RNAi molecule. In some embodiments, the RNA molecule is a siRNA molecule, a miRNA molecule, analogs thereof, precursors thereof, or a combination thereof. In some embodiments, the carrier provides for one or more of the following: stability for shortened duplexes, reduction or prevention of sense strand loading, reduction or prevention of seed region microRNA adverse side effects and reduction of non-specific immunoactivation. In some embodiments, the RNAi carrier is a di-lipid amino acid (DILA²). In some embodiments, the RNAi carrier is a Krebs Cycle analog. In some embodiments, the RNAi carrier is a Krebs Cycle analog and wherein the Krebs Cycle analog reduces or prevents cytotoxicity.

Disclosed herein, in certain embodiments, is a formulation, comprising: (a) an RNAi molecule comprising at least one of a locked nucleic acid (LNA), an unlocked nucleic acid (UNA), a bridged nucleic acid (BNA), a glycerol nucleic acid (GNA), or a combination thereof; and (b) an RNAi carrier. In some embodiments, the RNAi molecule is a siRNA molecule, a miRNA molecule, an analogue thereof, a precursor thereof, or combinations thereof. In some embodiments, the carrier provides for one or more of the following: stability for shortened duplexes, reduction or prevention of sense strand loading, reduction or prevention of seed region microRNA adverse side effects and reduction of non-specific immunoactivation. In some embodiments, the RNAi carrier is a di-lipid amino acid (DILA²). In some embodiments, the RNAi carrier is a Krebs Cycle analog. In some embodiments, the RNAi carrier is a Krebs Cycle analog and wherein the Krebs Cycle analog reduces or prevents cytotoxicity.

Disclosed herein, in certain embodiments, is a formulation, comprising: (a) an RNA molecule; and (b) an RNAi carrier. In some embodiments, the RNA molecule is an RNAi molecule. In some embodiments, the RNA molecule is a siRNA molecule, a miRNA molecule, analogs thereof, precursors thereof, or a combination thereof. In some embodiments, the RNA molecule comprises at least one of a locked nucleic acid (LNA), an unlocked nucleic acid (UNA), a bridged nucleic acid (BNA), a glycerol nucleic acid (GNA), or a combination thereof.

Disclosed herein, in certain embodiments, is a formulation, comprising: (a) an RNAi molecule comprising at least one of a locked nucleic acid (LNA), an unlocked nucleic acid (UNA), a bridged nucleic acid (BNA), a glycerol nucleic acid (GNA), or a combination thereof; and (b) a Krebs Cycle analog RNAi carrier. In some embodiments, the RNAi molecule is a siRNA molecule, a miRNA molecule, an analogue thereof, a precursor thereof, or combinations thereof.

Disclosed herein, in certain embodiments, is a formulation, comprising: (a) an RNAi molecule comprising at least one glycerol nucleic acid (GNA); and (b) a Krebs Cycle analog RNAi carrier.

Disclosed herein, in certain embodiments, is the use of an RNAi molecule disclosed herein or a formulation disclosed herein for the manufacture of a medicament for the treatment of cancer. In some embodiments, the RNAi molecule is a siRNA molecule, a miRNA molecule, an analogue thereof, a precursor thereof, or combinations thereof.

Disclosed herein, in certain embodiments, is the use of an RNAi molecule disclosed herein or a formulation disclosed herein for the manufacture of a medicament for inducing apoptosis of a circulating tumor cell (CTC). In some embodiments, the RNAi molecule is a siRNA molecule, a miRNA molecule, an analogue thereof, a precursor thereof, or combinations thereof.

Disclosed herein, in certain embodiments, is the use of an RNAi molecule disclosed herein or a formulation disclosed herein for the treatment of cancer. In some embodiments, the RNAi molecule is a siRNA molecule, a miRNA molecule, an analogue thereof, a precursor thereof, or combinations thereof. In some embodiments, the cancer is characterized by the presence of a primary tumor or a metastasis. In some embodiments, the cancer is breast cancer, a gastrointestinal cancer (such as a colon cancer), lung cancer or prostate cancer. In some embodiments, the RNAi molecule disclosed herein or a formulation disclosed herein is administered before, during, or immediately after surgery to remove a primary tumor or a metastasis. In some embodiments, the RNAi molecule disclosed herein or a formulation disclosed herein is locally administered at the site of the surgery. In some embodiments, the RNAi molecule disclosed herein or a formulation disclosed herein is administered in a time-release formulation. In some embodiments, the RNAi molecule disclosed herein or a formulation disclosed herein is administered by intravenous injection. In some embodiments, the RNAi molecule disclosed herein or a formulation disclosed herein exhibits reduced lipid-induced hepatic toxicity. In some embodiments, the RNAi molecule disclosed herein or a formulation disclosed herein reduces spread of the primary tumor or metastases.

Disclosed herein, in certain embodiments, is the use of an RNAi molecule disclosed herein or a formulation disclosed herein for inducing apoptosis of a circulating tumor cell (CTC). In some embodiments, the RNAi molecule is a siRNA molecule, a miRNA molecule, an analogue thereof, a precursor thereof, or combinations thereof. In some embodiments, the circulating tumor cell (CTC) is from a primary tumor or a metastasis. In some embodiments, the RNAi molecule disclosed herein or a formulation disclosed herein is administered in a time-release formulation. In some embodiments, the RNAi molecule disclosed herein or a formulation disclosed herein is administered by intravenous injection. In some embodiments, the RNAi molecule disclosed herein or a formulation disclosed herein is administered in a time-release formulation and by intravenous injection. In some embodiments, the RNAi molecule disclosed herein or a formulation disclosed herein exhibits reduced lipid-induced hepatic toxicity.

Disclosed herein, in certain embodiments, is the use of an RNAi molecule disclosed herein or a formulation disclosed herein for inhibiting cancerous and pre-cancerous gene expression of breast cancer-related genes and pre-cancerous-related genes. In some embodiments, the RNAi molecule is a siRNA molecule, a miRNA molecule, an analogue thereof, a precursor thereof, or combinations thereof. In some embodiments, the RNAi molecule disclosed herein or a formulation disclosed herein is administered to an individual presenting with premalignant or malignant breast duct epithelial cells in a breast duct. In some embodiments, the RNAi molecule disclosed herein or a formulation disclosed herein is administered locally the breast duct. In some embodiments, the formulation is administered in a time-release formulation.

### DETAILED DESCRIPTION OF THE INVENTION

Recent developments in the areas of gene therapy, antisense therapy and RNA interference therapy have created a need to develop efficient means of introducing nucleic acids into cells. Unfortunately, existing techniques for delivering nucleic acids to cells are limited by instability of the nucleic acids, poor efficiency and/or high toxicity of the delivery reagents. There is a need to provide for methods and formulations for effectively delivering double-stranded nucleic acids to cells to produce an effective therapy especially for delivering RNAi molecules (e.g., siRNA molecules, miRNA molecules, and analogues thereof) for RNA interference therapy.

Disclosed herein, in certain embodiments, is a formulation, comprising: (a) an RNA molecule comprising at least one of a locked nucleic acid (LNA), an unlocked nucleic acid (UNA), a bridged nucleic acid (BNA), a glycerol nucleic acid (GNA), or a combination thereof; and (b) an RNAi carrier. In some embodiments, the RNA molecule is an RNAi molecule. In some embodiments, the RNA molecule is a siRNA molecule, a miRNA molecule, analogs thereof, precursors thereof, or a combination thereof. In some embodiments, the carrier provides for one or more of the following: stability for shortened duplexes, reduction or prevention of sense strand loading, reduction or prevention of seed region microRNA adverse side effects and reduction of non-specific immunoactivation. In some embodiments, the RNAi carrier is a di-lipid amino acid (DILA²). In some embodiments, the RNAi carrier is a Krebs Cycle analog. In some embodiments, the RNAi carrier is a Krebs Cycle analog and wherein the Krebs Cycle analog reduces or prevents cytotoxicity. In some embodiments, the nucleic acid is a double stranded RNA.

Disclosed herein, in certain embodiments, is a formulation, comprising: (a) an RNAi molecule comprising at least one of a locked nucleic acid (LNA), an unlocked nucleic acid (UNA), a bridged nucleic acid (BNA), a glycerol nucleic acid (GNA), or a combination thereof; and (b) an RNAi carrier. In some embodiments, the RNAi molecule is a siRNA molecule, a miRNA molecule, an analogue thereof, a precursor thereof, or combinations thereof. In some embodiments, the carrier provides for one or more of the following: stability for shortened duplexes, reduction or prevention of sense strand loading, reduction or prevention of seed region microRNA adverse side effects and reduction of non-specific immunoactivation. In some embodiments, the RNAi carrier is a di-lipid amino acid (DILA²). In some embodiments, the RNAi carrier is a Krebs Cycle analog. In some embodiments, the RNAi carrier is a Krebs Cycle analog and wherein the Krebs Cycle analog reduces or prevents cytotoxicity. In some embodiments, the nucleic acid is a double stranded RNA.

Disclosed herein, in certain embodiments, is a formulation, comprising: (a) an RNA molecule; and (b) an RNAi carrier. In some embodiments, the RNA molecule is an RNAi molecule. In some embodiments, the RNA molecule is a siRNA molecule, a miRNA molecule, analogs thereof, precursors thereof, or a combination thereof. In some embodiments, the RNA molecule comprises at least one of a locked nucleic acid (LNA), an unlocked nucleic acid (UNA), a bridged nucleic acid (BNA), a glycerol nucleic acid (GNA), or a combination thereof.

Disclosed herein, in certain embodiments, is a formulation, comprising: (a) an RNAi molecule comprising at least one of a locked nucleic acid (LNA), an unlocked nucleic acid (UNA), a bridged nucleic acid (BNA), a glycerol nucleic acid (GNA), or a combination thereof; and (b) a Krebs Cycle analog RNAi carrier. In some embodiments, the RNAi molecule is a siRNA molecule, a miRNA molecule, an analogue thereof, a precursor thereof, or combinations thereof.

Disclosed herein, in certain embodiments, is a formulation, comprising: (a) an RNAi molecule comprising at least one glycerol nucleic acid (GNA); and (b) a Krebs Cycle analog RNAi carrier.

Disclosed herein, in certain embodiments, is the use of an RNAi molecule disclosed herein or a formulation disclosed herein for the manufacture of a medicament for the treatment of cancer. In some embodiments, the RNAi molecule is a siRNA molecule, a miRNA molecule, an analogue thereof, a precursor thereof, or combinations thereof.

Disclosed herein, in certain embodiments, is the use of an RNAi molecule disclosed herein or a formulation disclosed herein for the manufacture of a medicament for inducing apoptosis of a circulating tumor cell (CTC). In some embodiments, the RNAi molecule is a siRNA molecule, a miRNA molecule, an analogue thereof, a precursor thereof, or combinations thereof.

Disclosed herein, in certain embodiments, is the use of an RNAi molecule disclosed herein or a formulation disclosed herein for the treatment of cancer. In some embodiments, the RNAi molecule is a siRNA molecule, a miRNA molecule, an analogue thereof, a precursor thereof, or combinations thereof. In some embodiments, the cancer is characterized by the presence of a primary tumor or a metastasis. In some embodiments, the cancer is breast cancer, a gastrointestinal cancer (such as a colon cancer), lung cancer or prostate cancer. In some embodiments, the RNAi molecule disclosed herein or a formulation disclosed herein is administered before, during, or immediately after surgery to remove a primary tumor or a metastasis. In some embodiments, the RNAi molecule disclosed herein or a formulation disclosed herein is locally administered at the site of the surgery. In some embodiments, the RNAi molecule disclosed herein or a formulation disclosed herein is administered in a time-release formulation. In some embodiments, the RNAi molecule disclosed herein or a formulation disclosed herein is administered by intravenous injection. In some embodiments, the RNAi molecule disclosed herein or a formulation disclosed herein exhibits reduced lipid-induced hepatic toxicity. In some embodiments, the RNAi molecule disclosed herein or a formulation disclosed herein reduces spread of the primary tumor or metastases.

Disclosed herein, in certain embodiments, is the use of an RNAi molecule disclosed herein or a formulation disclosed herein for inducing apoptosis of a circulating tumor cell (CTC). In some embodiments, the RNAi molecule is a siRNA molecule, a miRNA molecule, an analogue thereof, a precursor thereof, or combinations thereof. In some embodiments, the circulating tumor cell (CTC) is from a primary tumor or a metastasis. In some embodiments, the RNAi molecule disclosed herein or a formulation disclosed herein is administered in a time-release formulation. In some embodiments, the RNAi molecule disclosed herein or a formulation disclosed herein is administered by intravenous injection. In some embodiments, the RNAi molecule disclosed herein or a formulation disclosed herein is administered in a time-release formulation and by intravenous injection. In some embodiments, the RNAi molecule disclosed herein or a formulation disclosed herein exhibits reduced lipid-induced hepatic toxicity.

Disclosed herein, in certain embodiments, is the use of an RNAi molecule disclosed herein or a formulation disclosed herein for inhibiting cancerous and pre-cancerous gene expression of breast cancer-related genes and pre-cancerous-related genes. In some embodiments, the RNAi molecule is a siRNA molecule, a miRNA molecule, an analogue thereof, a precursor thereof, or combinations thereof. In some embodiments, the RNAi molecule disclosed herein or a formulation disclosed herein is administered to an individual presenting with premalignant or malignant breast duct epithelial cells in a breast duct. In some embodiments, the RNAi molecule disclosed herein or a formulation disclosed herein is administered locally the breast duct. In some embodiments, the formulation is administered in a time-release formulation.

### CERTAIN DEFINITIONS

By “RNA” is meant a molecule comprising at least one ribonucleotide residue. By “ribonucleotide” is meant a nucleotide with a hydroxyl group at the 2′ position of a beta-D-ribo-furanose moiety. The term RNA includes, for example, double-stranded (ds) RNAs; single-stranded RNAs; and isolated RNAs such as partially purified RNA, essentially pure RNA, synthetic RNA, recombinantly produced RNA, as well as altered RNA that differ from naturally-occurring RNA by the addition, deletion, substitution and/or alteration of one or more nucleotides. Such alterations can include addition of non-nucleotide material, such as to the end(s) of the siRNA or internally, for example at one or more nucleotides of the RNA. Nucleotides in the RNA molecules described herein can also comprise non-standard nucleotides, such as non-naturally occurring nucleotides or chemically synthesized nucleotides or deoxynucleotides. These altered RNAs can be referred to as analogs or analogs of naturally-occurring RNA.

By “RNAi molecule” is meant an RNA molecule that induces RNAi. In some embodiments, the RNAi molecule is a dsRNA molecule that will generate a siRNA molecule or miRNA molecule following contact with Dicer (i.e., an RNAi molecule precursor). In some embodiments, the RNAi molecule is a siRNA duplex, a siRNA sense molecule, a siRNA anti-sense molecule, a miRNA duplex, a miRNA sense molecule, a miRNA anti-sense molecule, and analogues thereof.

By “sense region” is meant a nucleotide sequence of a siRNA molecule having complementarity to an anti-sense region of the siRNA molecule. In addition, the sense region of a siRNA molecule can comprise a nucleic acid sequence having homology with a target nucleic acid sequence.

By “anti-sense region” is meant a nucleotide sequence of a siRNA molecule having complementarity to a target nucleic acid sequence. In addition, the anti-sense region of a siRNA molecule can optionally comprise a nucleic acid sequence having complementarity to a sense region of the siRNA molecule.

The term “universal base” as used herein refers to nucleotide base analogs that form base pairs with each of the natural DNA/RNA bases with little discrimination between them. Non-limiting examples of universal bases include C-phenyl, C-naphthyl and other aromatic derivatives, inosine, azole carboxamides, and nitroazole derivatives such as 3-nitropyrrole, 4-nitroindole, 5-nitroindole, and 6-nitroindole as known in the art (see for example, Loakes, 2001, Nucleic Acids Research, 29:2437-2447).

The term “universal-binding nucleotide” as used herein refers to a nucleotide analog that is capable of forming a base-pairs with each of the natural DNA/RNA nucleotides with little discrimination between them. Non-limiting examples of universal-binding nucleotides include inosine, 1-beta-D-ribofuranosyl-5-nitroindole, and/or 1-beta-D-ribofuranosyl-3-nitropyrrole.

By “modulate gene expression” is meant that the expression of a target gene is upregulated or downregulated, which can include upregulation or down-regulation of mRNA levels present in a cell, or of mRNA translation, or of synthesis of protein or protein subunits, encoded by the target gene. Modulation of gene expression can be determined also be the presence, quantity, or activity of one or more proteins or protein subunits encoded by the target gene that is up regulated or down regulated, such that expression, level, or activity of the subject protein or subunit is greater than or less than that which is observed in the absence of the modulator (e.g., a siRNA).

By “inhibit”, “down-regulate”, “knockdown” or “reduce” expression, it is meant that the expression of the gene, or level of RNA molecules or equivalent RNA molecules encoding one or more proteins or protein subunits, or level or activity of one or more proteins or protein subunits encoded by a target gene, is reduced below that observed in the absence of the nucleic acid molecules (e.g., siRNA) described herein. In some embodiments, inhibition, down-regulation or reduction with a siRNA molecule is below that level observed in the presence of an inactive or attenuated molecule. In another embodiment, inhibition, down-regulation, or reduction with siRNA molecules is below that level observed in the presence of, for example, a siRNA molecule with scrambled sequence or with mismatches. In another embodiment, inhibition, down-regulation, or reduction of gene expression with a nucleic acid molecule described herein is greater in the presence of the nucleic acid molecule than in its absence.

Gene “silencing” refers to partial or complete loss-of-function through targeted inhibition of gene expression in a cell and may also be referred to as “knockdown”.

The phrase “inhibiting expression of a target gene” refers to the ability of an RNAi molecule (e.g., siRNA molecules, miRNA molecules, and analogues thereof) to initiate gene silencing of the target gene.

By “target nucleic acid” or “nucleic acid target” or “target RNA” or “RNA target” or “target DNA” or “DNA target” is meant any nucleic acid sequence whose expression or activity is to be modulated. The target nucleic acid can be DNA or RNA and is not limited single strand forms.

“Large double-stranded RNA” refers to any double-stranded RNA having a size greater than about 40 bp for example, larger than 100 bp or more particularly larger than 300 bp. The sequence of a large dsRNA may represent a segment of a mRNA or the entire mRNA. The maximum size of the large dsRNA is not limited herein. The double-stranded RNA may include modified bases where the modification may be to the phosphate sugar backbone or to the nucleoside. Such modifications may include a nitrogen or sulfur heteroatom or any other modification known in the art.

“Overlapping” refers to when two RNA fragments have sequences which overlap by a plurality of nucleotides on one strand, for example, where the plurality of nucleotides (nt) numbers as few as 2-5 nucleotides or by 5-10 nucleotides or more.

By “complementarity” is meant that a nucleic acid can form hydrogen bond(s) with another nucleic acid sequence by either traditional Watson-Crick or other non-traditional types. In reference to the nucleic molecules described herein, the binding free energy for a nucleic acid molecule with its complementary sequence is sufficient to allow the relevant function of the nucleic acid to proceed, e.g., RNAi activity. Determination of binding free energies for nucleic acid molecules is well known in the art (see, e.g., Turner et al., 1987, CSH Symp. Quant. Biol. LII pp. 123-133; Frier et al., 1986, Proc. Nat. Acad. Sci. USA 83:9373-9377; Turner et al., 1987, J. Am. Chem. Soc. 109:3783-3785). A percent complementarity indicates the percentage of contiguous residues in a nucleic acid molecule that can form hydrogen bonds (e.g., Watson-Crick base pairing) with a second nucleic acid sequence (e.g., 5, 6, 7, 8, 9, or 10 nucleotides out of a total of 10 nucleotides in the first oligonucleotide being based paired to a second nucleic acid sequence having 10 nucleotides represents 50%, 60%, 70%, 80%, 90%, and 100% complementary respectively). “Perfectly complementary” means that all the contiguous residues of a nucleic acid sequence will hydrogen bond with the same number of contiguous residues in a second nucleic acid sequence.

The term “pyrimidine” as used herein refers to conventional pyrimidines, including uracil and cytosine. In addition, the term pyrimidine is also contemplated to embrace “universal bases” that can be substituted within the formulations and methods described herein with a pyrimidine. As used herein the term “universal base” refers to nucleotide base analogs that form base pairs with each of the natural DNA/RNA bases with little discrimination between them. A universal base is thus interchangeable with all of the natural bases when substituted into an in an oligonucleotide duplex, typically yielding a duplex which primes DNA synthesis by a polymerase, directs incorporation of the 5′ triphosphate of each of the natural nucleosides opposite the universal base when copied by a polymerase, serves as a substrate for polymerases as the 5′-triphosphate, and is recognized by intracellular enzymes such that DNA containing the universal base can cloned. (Loakes et al., J. Mol Bio 270:426-435 (1997)). In all contexts herein where the term pyrimidine is employed, a universal base may thus be provided as an alternate, chemically modified base target for incorporating into a siRNA described herein. Non-limiting examples of universal bases include C-phenyl, C-naphthyl and other aromatic derivatives, inosine, azole carboxamides, and nitroazole derivatives such as 3-nitropyrrole, 4-nitroindole, 5-nitroindole, and 6-nitroindole as known in the art (see for example, Loakes, 2001, Nucleic Acids Research, 29:2437-2447).

By “subject” is meant an organism, which is a donor or recipient of explanted cells or the cells themselves. “Subject” also refers to an organism to which the nucleic acid molecules described herein can be administered. In some embodiments, a subject is a mammal or mammalian cells. In another embodiment, a subject is a human or human cells.

In this specification and the appended claims, the singular forms of “a”, “an” and “the” include plural reference unless the context clearly dictates otherwise.

### RNA Interference

Disclosed herein, in certain embodiments, is a formulation, comprising: (a) an RNA molecule comprising at least one of a locked nucleic acid (LNA), an unlocked nucleic acid (UNA), a bridged nucleic acid (BNA), a glycerol nucleic acid (GNA), or a combination thereof; and (b) an RNAi carrier. In some embodiments, the RNA molecule is an RNAi molecule. In some embodiments, the RNA molecule is a siRNA molecule, a miRNA molecule, analogs thereof, precursors thereof, or a combination thereof. In some embodiments, the carrier provides for one or more of the following: stability for shortened duplexes, reduction or prevention of sense strand loading, reduction or prevention of seed region microRNA adverse side effects and reduction of non-specific immunoactivation. In some embodiments, the RNAi carrier is a di-lipid amino acid (DILA²). In some embodiments, the RNAi carrier is a Krebs Cycle analog. In some embodiments, the RNAi carrier is a Krebs Cycle analog and wherein the Krebs Cycle analog reduces or prevents cytotoxicity. In some embodiments, the nucleic acid is a double stranded RNA.

Disclosed herein, in certain embodiments, is a formulation, comprising: (a) an RNAi molecule comprising at least one of a locked nucleic acid (LNA), an unlocked nucleic acid (UNA), a bridged nucleic acid (BNA), a glycerol nucleic acid (GNA), or a combination thereof; and (b) an RNAi carrier. In some embodiments, the RNAi molecule is a siRNA molecule, a miRNA molecule, an analogue thereof, a precursor thereof, or combinations thereof. In some embodiments, the carrier provides for one or more of the following: stability for shortened duplexes, reduction or prevention of sense strand loading, reduction or prevention of seed region microRNA adverse side effects and reduction of non-specific immunoactivation. In some embodiments, the RNAi carrier is a di-lipid amino acid (DILA²). In some embodiments, the RNAi carrier is a Krebs Cycle analog. In some embodiments, the RNAi carrier is a Krebs Cycle analog and wherein the Krebs Cycle analog reduces or prevents cytotoxicity. In some embodiments, the nucleic acid is a double stranded RNA.

Disclosed herein, in certain embodiments, is a formulation, comprising: (a) an RNA molecule; and (b) an RNAi carrier. In some embodiments, the RNA molecule is an RNAi molecule. In some embodiments, the RNA molecule is a siRNA molecule, a miRNA molecule, analogs thereof, precursors thereof, or a combination thereof. In some embodiments, the RNA molecule comprises at least one of a locked nucleic acid (LNA), an unlocked nucleic acid (UNA), a bridged nucleic acid (BNA), a glycerol nucleic acid (GNA), or a combination thereof.

Disclosed herein, in certain embodiments, is a formulation, comprising: (a) an RNAi molecule comprising at least one of a locked nucleic acid (LNA), an unlocked nucleic acid (UNA), a bridged nucleic acid (BNA), a glycerol nucleic acid (GNA), or a combination thereof; and (b) a Krebs Cycle analog RNAi carrier. In some embodiments, the RNAi molecule is a siRNA molecule, a miRNA molecule, an analogue thereof, a precursor thereof, or combinations thereof.

Disclosed herein, in certain embodiments, is a formulation, comprising: (a) an RNAi molecule comprising at least one glycerol nucleic acid (GNA); and (b) a Krebs Cycle analog RNAi carrier.

RNAi is an RNA-dependent gene silencing process that is controlled by the RNA-induced silencing complex (RISC) and is initiated by short double-stranded RNA molecules—microRNA (miRNA) and small interfering RNA (siRNA).

dsRNA initiates RNAi by activating the ribonuclease protein Dicer, which binds and cleaves double-stranded RNAs (dsRNAs) to produce double-stranded fragments of about 21-25 base pairs with a few unpaired overhang bases (about 2 to about 5 bp) on each end. These short double-stranded fragments are called small interfering RNAs (siRNAs) or micro RNAS. siRNA and miRNA molecules are then separated into single strands.

One of the two strands of each fragment, known as the guide strand, is then incorporated into the RNA-induced silencing complex (RISC). After integration into the RISC, the miRNA or siRNA molecule binds to a complementary sequence of mRNA. The binding of the guide strand allows Argonaute, the catalytic component of the RISC complex, to cleave the mRNA, thereby preventing it from being used as a translation template.

RNAi molecules useful for this invention may be targeted to various genes. In some embodiments, and RNAi molecule disclosed herein targets a gene (including mutations thereof and polymorphisms thereof) selected from: PI3K, MSH2, MLH1, PMS2, MSH6, PMS1, APC, prostate-cancer-gene-3 (PCA3), HPC1, PCAP, CAPB, HPC2, HPC20, HPCX, MSR1, ELAC2, e.g., RNASEL/HPC1, ELAC2/HPC2, SR-A/MSR1, CHEK2, BRCA2, PON1, OGG1, MIC-1, TLR4, and PTEN), BRCA1, BRCA2, CDH1, PTEN, STK11, TP53, AR, ATM, BARD1, BRIP1, CHEK2, DIRAS3, ERBB2, NBN, PALB2, RAD50, RAD51, or combinations thereof. Examples of additional human genes suitable as targets include TNF, FLT1, the VEGF family, the ERBB family, the PDGFR family, BCR-ABL, and the MAPK family, among others. Examples of human genes suitable as targets and nucleic acid sequences thereto include those disclosed in PCT/U.S.08/55333, PCT/US08/55339, PCT/US08/55340, PCT/US08/55341, PCT/US08/55350, PCT/US08/55353, PCT/US08/55356, PCT/US08/55357, PCT/US08/55360, PCT/US08/55362, PCT/US08/55365, PCT/US08/55366, PCT/US08/55369, PCT/US08/55370, PCT/US08/55371, PCT/US08/55372, PCT/US08/55373, PCT/US08/55374, PCT/US08/55375, PCT/US08/55376, PCT/US08/55377, PCT/US08/55378, PCT/US08/55380, PCT/US08/55381, PCT/US08/55382, PCT/US08/55383, PCT/US08/55385, PCT/US08/55386, PCT/US08/55505, PCT/US08/55511, PCT/US08/55515, PCT/US08/55516, PCT/US08/55519, PCT/US08/55524, PCT/US08/55526, PCT/US08/55527, PCT/US08/55532, PCT/US08/55533, PCT/US08/55542, PCT/US08/55548, PCT/US08/55550, PCT/US08/55551, PCT/US08/55554, PCT/US08/55556, PCT/US08/55560, PCT/US08/55563, PCT/US08/55597, PCT/US08/55599, PCT/US08/55601, PCT/US08/55603, PCT/US08/55604, PCT/US08/55606, PCT/US08/55608, PCT/US08/55611, PCT/US08/55612, PCT/US08/55615, PCT/US08/55618, PCT/US08/55622, PCT/US08/55625, PCT/US08/55627, PCT/US08/55631, PCT/US08/55635, PCT/US08/55644, PCT/US08/55649, PCT/US08/55651, PCT/US08/55662, PCT/US08/55672, PCT/US08/55676, PCT/US08/55678, PCT/US08/55695, PCT/US08/55697, PCT/US08/55698, PCT/US08/55701, PCT/US08/55704, PCT/US08/55708, PCT/US08/55709, and PCT/US08/55711.

### Generation of RNAi Molecules

In some embodiments, a double stranded RNA (dsRNA) molecule with sequences complementary to a target is generated. The synthesis of a dsRNA molecule comprises: (a) synthesis of two complementary strands of the RNAi molecule; and (b) annealing the two complementary strands together under conditions suitable to obtain a double-stranded RNA molecule. In another embodiment, synthesis of the two complementary strands of the RNA molecule is by solid phase oligonucleotide synthesis. In yet another embodiment, synthesis of the two complementary strands of the RNA molecule is by solid phase tandem oligonucleotide synthesis. In some embodiments, a nucleic acid molecule described herein is synthesized separately and joined together post-synthetically, for example, by ligation or by hybridization following synthesis and/or deprotection. Oligonucleotides (e.g., certain modified oligonucleotides or portions of oligonucleotides lacking ribonucleotides) are synthesized using any suitable method.

RNAi constructs can be purified by gel electrophoresis or can be purified by high pressure liquid chromatography.

### Design of RNAi Molecules

In some embodiments, an RNAi molecule (e.g., siRNA molecules, miRNA molecules, and analogues thereof) is about 20-25 bp. In some embodiments, the 20-25 bp RNAi molecule (e.g., siRNA molecules, miRNA molecules, and analogues thereof) has 2-5 bp overhangs on the 3′ end of each strand, and a 5′ phosphate terminus and a 3′ hydroxyl terminus. In some embodiments, the 20-25 bp RNAi molecule (e.g., siRNA molecules, miRNA molecules, and analogues thereof) has blunt ends.

In some embodiments, an RNAi molecule (e.g., siRNA molecules, miRNA molecules, and analogues thereof) is assembled from two separate oligonucleotides, where one strand is the sense strand and the other is the anti-sense strand, wherein the anti-sense and sense strands are self-complementary (i.e. each strand comprises nucleotide sequence that is complementary to nucleotide sequence in the other strand; such as where the anti-sense strand and sense strand form a duplex or double stranded structure, for example wherein the double stranded region is about 19 base pairs). In some embodiments, the anti-sense strand of an RNAi molecule (e.g., siRNA molecules, miRNA molecules, and analogues thereof) comprises a nucleotide sequence that is complementary to a nucleotide sequence in a target nucleic acid molecule or a portion thereof, and the sense strand comprises a nucleotide sequence corresponding to the target nucleic acid sequence or a portion thereof. In some embodiments, an RNAi molecule (e.g., siRNA molecules, miRNA molecules, and analogues thereof) is assembled from a single oligonucleotide, where the self-complementary sense and anti-sense regions of the RNAi molecule (e.g., siRNA molecules, miRNA molecules, and analogues thereof) are linked by means of a nucleic acid-based or non-nucleic acid-based linker(s).

In some embodiments, an RNAi molecule (e.g., siRNA molecules, miRNA molecules, and analogues thereof) comprises a single stranded polynucleotide having nucleotide sequence complementary to nucleotide sequence in a target nucleic acid molecule or a portion thereof (for example, where such RNAi molecule (e.g., siRNA molecules, miRNA molecules, and analogues thereof) does not require the presence within the RNAi molecule (e.g., siRNA molecules, miRNA molecules, and analogues thereof) of nucleotide sequence corresponding to the target nucleic acid sequence or a portion thereof), wherein the single stranded polynucleotide further comprises a terminal phosphate group, such as a 5′-phosphate, or 5′,3′-diphosphate.

In other embodiments, an RNAi molecule (e.g., siRNA molecules, miRNA molecules, and analogues thereof) comprises separate sense and anti-sense sequences or regions, wherein the sense and anti-sense regions are covalently linked by nucleotide or non-nucleotide linker molecules, or are alternately non-covalently linked by ionic interactions, hydrogen bonding, van der Waals interactions, hydrophobic interactions, and/or stacking interactions.

21 nucleotide RNAi molecule (e.g., siRNA molecules, miRNA molecules, and analogues thereof) are most active when containing two nucleotide 3′-overhangs. Furthermore, complete substitution of one or both RNAi molecule (e.g., siRNA molecules, miRNA molecules, and analogues thereof) strands with 2′-deoxy (2′-H) or 2′-O-methyl nucleotides abolishes RNAi molecule (e.g., siRNA molecules, miRNA molecules, and analogues thereof) activity, whereas substitution of the 3′-terminal RNAi molecule (e.g., siRNA molecules, miRNA molecules, and analogues thereof) overhang nucleotides with deoxy nucleotides (2′-H) has been reported to be tolerated.

Replacing the 3′-overhanging segments of a 21-mer RNAi molecule (e.g., siRNA molecules, miRNA molecules, and analogues thereof) having 2 nucleotide 3′ overhangs with deoxyribonucleotides does not have an adverse effect on RNAi molecule (e.g., siRNA molecules, miRNA molecules, and analogues thereof) activity. In certain instances, replacing up to 4 nucleotides on each end of the RNAi molecule (e.g., siRNA molecules, miRNA molecules, and analogues thereof) with deoxyribonucleotides is well tolerated whereas complete substitution with deoxyribonucleotides results in no RNAi molecule (e.g., siRNA molecules, miRNA molecules, and analogues thereof) activity.

The terminal structure of RNAi molecules described herein is either blunt or cohesive (overhanging). In some embodiments, the cohesive (overhanging) end structure is a 3′ overhang or a 5′ overhang. In some embodiments, the number of overhanging nucleotides is any length as long as the overhang does not impair gene silencing activity. In some embodiments, an overhang sequence is not complementary (anti-sense) or identical (sense) to the target gene sequence. In some embodiments, the overhang sequence contains low molecular weight structures (for example a natural RNA molecule such as tRNA, rRNA or tumor or CTC RNA, or an artificial RNA molecule).

The total length of RNAi molecules having cohesive end structure is expressed as the sum of the length of the paired double-stranded portion and that of a pair comprising overhanging single-strands at both ends. For example, in the exemplary case of a 19 bp double-stranded RNA with 4 nucleotide overhangs at both ends, the total length is expressed as 23 bp.

In some embodiments, the terminal structure of an RNAi molecule (e.g., siRNA molecules, miRNA molecules, and analogues thereof) has a stem-loop structure in which ends of one side of the double-stranded nucleic acid are connected by a linker nucleic acid, e.g., a linker RNA. In some embodiments, the length of the double-stranded region (stem-loop portion) is 15 to 49 bp, often 15 to 35 bp, and more commonly about 21 to 30 bp long.

In some embodiments, an RNAi molecules is a polynucleotide with a duplex, asymmetric duplex, hairpin or asymmetric hairpin secondary structure, having self-complementary sense and anti-sense regions, wherein the anti-sense region comprises a nucleotide sequence that is complementary to a nucleotide sequence in a separate target nucleic acid molecule or a portion thereof, and the sense region comprises a nucleotide sequence corresponding to the target nucleic acid sequence or a portion thereof.

In some embodiments, an RNAi molecule (e.g., siRNA molecules, miRNA molecules, and analogues thereof) comprises a circular nucleic acid molecule, wherein the RNAi molecule (e.g., siRNA molecules, miRNA molecules, and analogues thereof) is about 38 to about 70 (e.g., about 38, 40, 45, 50, 55, 60, 65, or 70) nucleotides in length having about 18 to about 23 (e.g., about 18, 19, 20, 21, 22, or 23) base pairs wherein the circular oligonucleotide forms a dumbbell shaped structure having about 19 base pairs and 2 loops.

In some embodiments, a circular RNAi molecule (e.g., siRNA molecules, miRNA molecules, and analogues thereof) contains two loop motifs, wherein one or both loop portions of the RNAi molecule (e.g., siRNA molecules, miRNA molecules, and analogues thereof) is biodegradable. In some embodiments, degradation of the loop portions of a circular RNAi molecule (e.g., siRNA molecules, miRNA molecules, and analogues thereof) generates a double-stranded RNAi molecule (e.g., siRNA molecules, miRNA molecules, and analogues thereof) with 3′-terminal overhangs, such as 3′-terminal nucleotide overhangs comprising about 2 nucleotides.

The sense strand of a double stranded RNAi molecule (e.g., siRNA molecules, miRNA molecules, and analogues thereof) may have a terminal cap moiety such as an inverted deoxybasic moiety, at the 3′-end, 5′-end, or both 3′ and 5′-ends of the sense strand.

In some embodiments, an RNAi molecule (e.g., siRNA molecules, miRNA molecules, and analogues thereof) is modified. In some embodiments, a modified RNAi molecule (e.g., siRNA molecules, miRNA molecules, and analogues thereof) has properties or characteristics similar to naturally occurring ribonucleotides.

In some embodiments, the phosphate backbone of an RNAi molecule (e.g., siRNA molecules, miRNA molecules, and analogues thereof) is modified. Modifications include, but are not limited to, one or more phosphorothioate, phosphorodithioate, methylphosphonate, phosphotriester, morpholino, amidate carbamate, carboxymethyl, acetamidate, polyamide, sulfonate, sulfonamide, sulfamate, formacetal, thioformacetal, and/or alkylsilyl, substitutions.

In some embodiments, the 3′-terminal nucleotide overhangs of an RNAi molecule (e.g., siRNA molecules, miRNA molecules, and analogues thereof) comprise ribonucleotides or deoxyribonucleotides that are chemically-modified at a nucleic acid sugar, base, or backbone. In some embodiments, the 3′-terminal nucleotide overhangs comprises one or more universal base ribonucleotides. In some embodiments, the 3′-terminal nucleotide overhangs comprises one or more acyclic nucleotides.

In some embodiments, some or all of the ribose uracils of an RNAi molecule (e.g., siRNA molecules, miRNA molecules, and analogues thereof) are replaced with ribose thymine. In some embodiments, the stability of double-stranded RNA is greatly increased and is less susceptible to degradation by RNAses when ribose uracils are change to ribose thymine in both the sense and anti-sense strands of the RNA.

In some embodiments, modification of RNAi molecules (e.g., the introduction of chemically-modified nucleotides into nucleic acid molecules) result in RNAi molecules with increased in vivo stability and bioavailability. For example, the use of chemically-modified nucleic acid molecules can enable a lower dose of a particular nucleic acid molecule for a given therapeutic effect since chemically-modified nucleic acid molecules tend to have a longer half-life in serum. Furthermore, certain chemical modifications can improve the bioavailability of nucleic acid molecules by targeting particular cells or tissues and/or improving cellular uptake of the nucleic acid molecule. Therefore, even if the activity of a chemically-modified nucleic acid molecule is reduced as compared to a native nucleic acid molecule, for example, when compared to an all-RNA nucleic acid molecule, the overall activity of the modified nucleic acid molecule can be greater than that of the native molecule due to improved stability and/or delivery of the molecule. Unlike native non-modified RNAi molecule, chemically-modified RNAi molecule (e.g., siRNA molecules, miRNA molecules, and analogues thereof) can also minimize the possibility of activating interferon activity in humans.

In some embodiments, an RNAi molecule (e.g., siRNA molecules, miRNA molecules, and analogues thereof) is modified to prevent degradation by serum ribonucleases. In some embodiments, sugar, base and phosphate modifications increase the nuclease stability and efficacy of an RNAi molecule. For example, oligonucleotides are modified to enhance stability and/or enhance biological activity by modification with nuclease resistant groups, for example, 2′-amino, 2′-C-allyl, 2′-fluoro, 2′-O-methyl, 2′-O-allyl, 2′-H, nucleotide base modifications. Other modifications that can increase serum stability include, but are not limited to, phosphorothioate internucleotide linkages, 2′-deoxyribonucleotides, 2′-O-methyl ribonucleotides, 2′-deoxy-2′-fluoro ribonucleotides, “universal base” nucleotides, “acyclic” nucleotides, 5-C-methyl nucleotides, and terminal glyceryl and/or inverted deoxy abasic residue incorporation

In some embodiments, modification of RNAi molecule (e.g., siRNA molecules, miRNA molecules, and analogues thereof) reduces “off-target effects” of the RNAi molecule (e.g., siRNA molecules, miRNA molecules, and analogues thereof) when it is contacted with a biological sample (e.g., when introduced into a target eukaryotic cell having specific, and non-specific mRNA species present as potential specific and non-specific targets).

In some embodiments, modification of RNAi molecule (e.g., siRNA molecules, miRNA molecules, and analogues thereof) reduces interferon activation by the RNAi molecule (e.g., siRNA molecules, miRNA molecules, and analogues thereof) when the RNAi molecule (e.g., siRNA molecules, miRNA molecules, and analogues thereof) is contacted with a biological sample, e.g., when introduced into a eukaryotic cell.

In some embodiments, incorporation of a multiply-modified polynucleotide into an RNAi molecule (e.g., siRNA molecules, miRNA molecules, and analogues thereof) increases resistance of the RNAi molecule (e.g., siRNA molecules, miRNA molecules, and analogues thereof) to enzymatic degradation, particularly exonucleolytic degradation, including 5′ exonucleolytic and/or 3′ exonucleolytic degradation.

In some embodiments, an RNAi molecule (e.g., siRNA molecules, miRNA molecules, and analogues thereof) is modified by the incorporation of one or more multiply-modified ribonucleotide(s). In some embodiments, multiply-modified ribonucleotide are incorporated at the 3′ and/or 5′ end of one or both strands of the RNAi molecule. In some embodiments, multiply-modified ribonucleotides are not incorporated at internal positions in the RNAi molecule. Typically, fewer than 10, often fewer than 8, more often fewer than 6, and usually less then 2-4 multiply-modified ribonucleotides are incorporated internally within a sense or anti-sense strand, or among both strands collectively, in the modified RNAi molecule.

In addition to increasing resistance of the modified RNAi molecules to exonucleolytic degradation, in some embodiments, the incorporation of one or more multiply-modified ribonucleotide(s) renders an RNAi molecule (e.g., siRNA molecules, miRNA molecules, and analogues thereof) more resistant to other enzymatic and/or chemical degradation processes, and thus more stable and bioavailable than otherwise identical RNAi molecules that do not include the modified ribonucleotide(s).

In addition to increasing stability of modified RNAi molecules, in some embodiments, incorporation of one or more multiply-modified polynucleotides in an RNAi molecule (e.g., siRNA molecules, miRNA molecules, and analogues thereof) yields additional desired functional results, including increasing a melting point of a modified RNAi molecule (e.g., siRNA molecules, miRNA molecules, and analogues thereof) compared to a corresponding, non-modified RNAi molecule. By increasing an RNAi molecule (e.g., siRNA molecules, miRNA molecules, and analogues thereof) melting point, the subject modifications block or reduce the occurrence or extent of partial dehybridization of the modified RNAi molecule, thereby increasing the stability of the modified RNAi molecule.

In some embodiments, a multiple modification is introduced into one or more pyrimidines, or into any combination and up to all pyrimidines present in one or both strands of the RNAi molecule.

In some embodiments, an RNAi molecule (e.g., siRNA molecules, miRNA molecules, and analogues thereof) comprises one or more universal-binding nucleotide(s). A universal-binding nucleotide is a nucleotide that is able to form a hydrogen bonded nucleotide pair with more than one nucleotide type. Universal-binding nucleotides include, but are not limited to, inosine (I), 1-beta-D-ribofuranosyl-5-nitroindole, and 1-beta-D-ribofuranosyl-3-nitropyrrole. Inosine is a universal-binding nucleotide that pairs with an adenine (A), uracil (U), and cytosine (C) nucleotide, but not guanine (G).

In some embodiments, an RNAi molecule (e.g., siRNA molecules, miRNA molecules, and analogues thereof) comprises at least one or more universal-binding nucleotides, wherein the at least one or more universal-binding nucleotides. In some embodiments, an RNAi molecule (e.g., siRNA molecules, miRNA molecules, and analogues thereof) comprises between about 1 universal-binding nucleotide and about 10 universal-binding nucleotides.

In some embodiments, an RNAi molecule (e.g., siRNA molecules, miRNA molecules, and analogues thereof) comprises one or more universal-binding nucleotide(s) in the first, second and/or third position in the anti-codon of the anti-sense strand of the RNAi molecule.

In some embodiments, the isoleucine anti-codon UAU is modified such that the third-position uracil (U) nucleotide is substituted with the universal-binding nucleotide inosine (I) to create the anti-codon UAI. This modified anti-codon UAI increases the specific-binding capacity of the RNAi molecule (e.g., siRNA molecules, miRNA molecules, and analogues thereof) and thus permits the RNAi molecule (e.g., siRNA molecules, miRNA molecules, and analogues thereof) to pair with mRNAs having any one of AUA, UUA, and CUA in the corresponding position of the coding strand thereby expanding the number of available RNA degradation targets to which the RNAi molecule (e.g., siRNA molecules, miRNA molecules, and analogues thereof) specifically binds.

In some embodiments, the anti-codon AUA is modified by substituting a universal-binding nucleotide in the third or second position of the anti-codon such that the anti-codon(s) represented by UAI (third position substitution) or UIU (second position substitution) to generate RNAi molecule (e.g., siRNA molecules, miRNA molecules, and analogues thereof) that are capable of specifically binding to AUA, CUA and UUA and AAA, ACA and AUA.

It will be understood that, regardless of the position at which the one or more universal-binding nucleotide is substituted, the RNAi molecule (e.g., siRNA molecules, miRNA molecules, and analogues thereof) is capable of binding to a target gene and one or more variant(s) thereof thereby facilitating the degradation of the target gene and/or variant thereof via a RISC complex. Thus, an RNAi molecule (e.g., siRNA molecules, miRNA molecules, and analogues thereof) is suitable for introduction into cells to mediate targeted post-transcriptional gene silencing of a target gene and/or variants thereof. When an RNAi molecule (e.g., siRNA molecules, miRNA molecules, and analogues thereof) is inserted into a cell, the RNAi molecule (e.g., siRNA molecules, miRNA molecules, and analogues thereof) duplex is then unwound, and the anti-sense strand of the duplex is loaded into an assembly of proteins to form the RNA-induced silencing complex (RISC).

In some embodiments, an RNAi molecule (e.g., siRNA molecules, miRNA molecules, and analogues thereof) is modified with phosphorothioate internucleotide linkages. In some embodiments, the anti-sense region of an RNAi molecule (e.g., siRNA molecules, miRNA molecules, and analogues thereof) comprises a phosphorothioate internucleotide linkage at the 3′-end of the anti-sense region. In some embodiments, the anti-sense region comprises about one to about five phosphorothioate internucleotide linkages at the 5′-end of the anti-sense region. In some embodiments, both strands of an RNAi molecule (e.g., siRNA molecules, miRNA molecules, and analogues thereof) have about 1, 2, 3, 4, 5, 6, 7, 8 or more phosphorothioate internucleotide linkages. In some embodiments, one strand of an RNAi molecule (e.g., siRNA molecules, miRNA molecules, and analogues thereof) has about 1, 2, 3, 4, 5, 6, 7, 8 or more phosphorothioate internucleotide linkages. In some embodiments, one or both strands of the RNAi molecule (e.g., siRNA molecules, miRNA molecules, and analogues thereof) comprises one or more phosphorothioate internucleotide linkages at the 3′-end, the 5′-end, or both of the 3′- and 5′-ends. For example, an exemplary RNAi molecule (e.g., siRNA molecules, miRNA molecules, and analogues thereof) can comprise about 1 to about 5 or more (e.g., about 1, 2, 3, 4, 5, or more) consecutive phosphorothioate internucleotide linkages at the 5′-end of the sense strand, the anti-sense strand, or both strands. In another non-limiting example, an exemplary RNAi molecule (e.g., siRNA molecules, miRNA molecules, and analogues thereof) can comprise one or more (e.g., about 1, 2, 3, 4, 5, 6, 7, 8, 9, 10, or more) pyrimidine phosphorothioate internucleotide linkages in the sense strand, the anti-sense strand, or both strands. In yet another non-limiting example, an exemplary RNAi molecule (e.g., siRNA molecules, miRNA molecules, and analogues thereof) can comprise one or more (e.g., about 1, 2, 3, 4, 5, 6, 7, 8, 9, 10, or more) purine phosphorothioate internucleotide linkages in the sense strand, the anti-sense strand, or both strands.

In some embodiments, an RNAi molecule (e.g., siRNA molecules, miRNA molecules, and analogues thereof) is comprised of a nucleotide, non-nucleotide, or mixed nucleotide/non-nucleotide linker that joins the sense region of the RNAi to the anti-sense region of the RNAi. In some embodiments, a nucleotide linker can be a linker of >2 nucleotides in length, for example about 3, 4, 5, 6, 7, 8, 9, or 10 nucleotides in length.

In another embodiment, the nucleotide linker can be a nucleic acid aptamer. By “aptamer” or “nucleic acid aptamer” as used herein is meant a nucleic acid molecule that binds specifically to a target molecule wherein the nucleic acid molecule has sequence that comprises a sequence recognized by the target molecule in its natural setting. Alternately, an aptamer can be a nucleic acid molecule that binds to a target molecule where the target molecule does not naturally bind to a nucleic acid. The target molecule can be any molecule of interest.

In some embodiments, a non-nucleotide linker is comprised of an abasic nucleotide, polyether, polyamine, polyamide, peptide, carbohydrate, lipid, polyhydrocarbon, or other polymeric compounds (e.g. polyethylene glycols such as those having between 2 and 100 ethylene glycol units).

When linker segments are employed, there is no particular limitation in the length of the linker as long as it does not hinder pairing of the stem portion. For example, for stable pairing of the stem portion and suppression of recombination between DNAs coding for this portion, the linker portion may have a clover-leaf tRNA structure. Even if the linker has a length that would hinder pairing of the stem portion, it is possible, for example, to construct the linker portion to include introns so that the introns are excised during processing of a precursor RNA into mature RNA, thereby allowing pairing of the stem portion. In the case of a stem-loop RNAi, either end (head or tail) of RNA with no loop structure may have a low molecular weight RNA. As described above, these low molecular weight RNAs may include a natural RNA molecule, such as tRNA, rRNA or tumor or CTC RNA, or an artificial RNA molecule.

In some embodiments, the RNAi molecule (e.g., siRNA molecules, miRNA molecules, and analogues thereof) comprises at least one unlocked nucleotide. In some embodiments, the RNAi molecule (e.g., siRNA molecules, miRNA molecules, and analogues thereof) comprises at least one nucleotide in which the bond between the C2′ and C3′ atoms has been cleaved.

In some embodiments, the RNAi molecule (e.g., siRNA molecules, miRNA molecules, and analogues thereof) comprises at least one bridged or locked nucleotide. In some embodiments, a methylene bridge locks the nucleotide. In some embodiments, an RNAi molecule (e.g., siRNA molecules, miRNA molecules, and analogues thereof) comprises at least one nucleotide comprising a methylene bridge between the 2′ oxygen and 4′ carbon. In some embodiments, the ribose of at least one nucleotide is locked in the North conformation.

In some embodiments, the RNAi molecule (e.g., siRNA molecules, miRNA molecules, and analogues thereof) comprises at least one glycerol nucleotide. In some embodiments, the ribose backbone of a nucleotide is replaced with a glycerol.

### Selection of RNAi Molecules

In some embodiments, an RNAi molecule (e.g., siRNA molecules, miRNA molecules, and analogues thereof) disclosed herein is capable of specifically binding to desired gene target variants while being incapable of specifically binding to non-desired gene target variants.

In some embodiments, an RNAi molecule (e.g., siRNA molecules, miRNA molecules, and analogues thereof) is selected for use in a method disclosed herein based on predictions of the stability of molecule. In some embodiments, a prediction of stability is achieved by employing a theoretical melting curve wherein a higher theoretical melting curve indicates an increase in the molecule's stability and a concomitant decrease in cytotoxic effects. In some embodiments, stability of an RNAi molecule (e.g., siRNA molecules, miRNA molecules, and analogues thereof) is determined empirically by measuring the hybridization of a single modified RNA strand containing one or more universal-binding nucleotide(s) to a complementary target gene within, for example, a polynucleotide array. In some embodiments, the melting temperature (i.e., the Tm value) for each modified RNA and complementary RNA immobilized on the array is determined and, from this Tm value, the relative stability of the modified RNA pairing with a complementary RNA molecule determined.

In some embodiments, an RNAi molecule (e.g., siRNA molecules, miRNA molecules, and analogues thereof) is selected for use in a method disclosed herein based on “off-target” profiling whereby one or more RNAi molecules is administered to a cell(s), either in vivo or in vitro, and total mRNA is collected, and used to probe a microarray comprising oligonucleotides having one or more nucleotide sequence from a panel of known genes, including non-target genes. The “off-target” profile of the modified RNAi molecule (e.g., siRNA molecules, miRNA molecules, and analogues thereof) is quantified by determining the number of non-target genes having reduced expression levels in the presence of the RNAi molecule. The existence of “off target” binding indicates an RNAi molecule (e.g., siRNA molecules, miRNA molecules, and analogues thereof) that is capable of specifically binding to one or more non-target gene. Ideally, an RNAi molecule (e.g., siRNA molecules, miRNA molecules, and analogues thereof) applicable to therapeutic use will exhibit a high Tm value while exhibiting little or no “off-target” binding.

In some embodiments, an RNAi molecule (e.g., siRNA molecules, miRNA molecules, and analogues thereof) is selected for use in a method disclosed herein by use of a report gene assay. In some embodiments, a reporter gene construct comprises a constitutive promoter, for example the cytomegalovirus (CMV) or phosphoglycerate kinase (PGK) promoter, operably fused to, and capable of modulating the expression of, one or more reporter gene such as, for example, a luciferase gene, a chloramphenicol (CAT) gene, and/or a β-galactosidase gene, which, in turn, is operably fused in-frame with an oligonucleotide (typically between about 15 base-pairs and about 40 base-pairs, more typically between about 19 base-pairs and about 30 base-pairs, most typically 20, 21, 22, 23, 24, 25, 26, 27, 28, or 29 base-pairs) that contains a target sequence for the one or more RNAi molecules. In some embodiments, individual reporter gene expression constructs are co-transfected with one or more RNAi molecules. In some embodiments, the capacity of a given RNAi molecule (e.g., siRNA molecules, miRNA molecules, and analogues thereof) to reduce the expression level of each of the contemplated gene variants is determined by comparing the measured reporter gene activity from cells transfected with and without the modified RNAi molecule.

In some embodiments, an RNAi molecule (e.g., siRNA molecules, miRNA molecules, and analogues thereof) is selected for use in a method disclosed herein by assaying its ability to specifically bind to an mRNA, such as an mRNA expressed by a target tumor cell or circulating tumor cell (CTC).

In some embodiments, the assay comprises (a) selecting a target gene, wherein the target gene is a target tumor gene, for RNAi; and (b) administering one or more RNAi molecules to a cell expressing mRNA from the target tumor gene. In some embodiments, an RNAi molecule (e.g., siRNA molecules, miRNA molecules, and analogues thereof) is selected if it inhibits or reduces expression of the target tumor gene.

### Methods of Use

Disclosed herein, in certain embodiments, is the use of an RNAi molecule disclosed herein or a formulation disclosed herein for the manufacture of a medicament for the treatment of cancer. In some embodiments, the RNAi molecule is a siRNA molecule, a miRNA molecule, an analogue thereof, a precursor thereof, or combinations thereof.

Disclosed herein, in certain embodiments, is the use of an RNAi molecule disclosed herein or a formulation disclosed herein for the manufacture of a medicament for inducing apoptosis of a circulating tumor cell (CTC). In some embodiments, the RNAi molecule is a siRNA molecule, a miRNA molecule, an analogue thereof, a precursor thereof, or combinations thereof.

Disclosed herein, in certain embodiments, is the use of an RNAi molecule disclosed herein or a formulation disclosed herein for the treatment of cancer. In some embodiments, the RNAi molecule is a siRNA molecule, a miRNA molecule, an analogue thereof, a precursor thereof, or combinations thereof. In some embodiments, the cancer is characterized by the presence of a primary tumor or a metastasis. In some embodiments, the cancer is breast cancer, a gastrointestinal cancer (such as a colon cancer), lung cancer or prostate cancer. In some embodiments, the RNAi molecule disclosed herein or a formulation disclosed herein is administered before, during, or immediately after surgery to remove a primary tumor or a metastasis. In some embodiments, the RNAi molecule disclosed herein or a formulation disclosed herein is locally administered at the site of the surgery. In some embodiments, the RNAi molecule disclosed herein or a formulation disclosed herein is administered in a time-release formulation. In some embodiments, the RNAi molecule disclosed herein or a formulation disclosed herein is administered by intravenous injection. In some embodiments, the RNAi molecule disclosed herein or a formulation disclosed herein exhibits reduced lipid-induced hepatic toxicity. In some embodiments, the RNAi molecule disclosed herein or a formulation disclosed herein reduces spread of the primary tumor or metastases.

Disclosed herein, in certain embodiments, is the use of an RNAi molecule disclosed herein or a formulation disclosed herein for inducing apoptosis of a circulating tumor cell (CTC). In some embodiments, the RNAi molecule is a siRNA molecule, a miRNA molecule, an analogue thereof, a precursor thereof, or combinations thereof. In some embodiments, the circulating tumor cell (CTC) is from a primary tumor or a metastasis. In some embodiments, the RNAi molecule disclosed herein or a formulation disclosed herein is administered in a time-release formulation. In some embodiments, the RNAi molecule disclosed herein or a formulation disclosed herein is administered by intravenous injection. In some embodiments, the RNAi molecule disclosed herein or a formulation disclosed herein is administered in a time-release formulation and by intravenous injection. In some embodiments, the RNAi molecule disclosed herein or a formulation disclosed herein exhibits reduced lipid-induced hepatic toxicity.

Disclosed herein, in certain embodiments, is the use of an RNAi molecule disclosed herein or a formulation disclosed herein for inhibiting cancerous and pre-cancerous gene expression of breast cancer-related genes and pre-cancerous-related genes. In some embodiments, the RNAi molecule is a siRNA molecule, a miRNA molecule, an analogue thereof, a precursor thereof, or combinations thereof. In some embodiments, the RNAi molecule disclosed herein or a formulation disclosed herein is administered to an individual presenting with premalignant or malignant breast duct epithelial cells in a breast duct. In some embodiments, the RNAi molecule disclosed herein or a formulation disclosed herein is administered locally the breast duct. In some embodiments, the formulation is administered in a time-release formulation.

In some embodiments, the cancer is early stage cancer, non-metastatic cancer, advanced cancer, locally advanced cancer, metastatic cancer, cancer in remission, cancer that is substantially refractory to chemotherapy or cancer that is substantially refractory to hormone therapy. In some embodiments, the cancer is metastatic cancer. In some embodiments, the cancer is a solid tumor.

In some embodiments, the cancer is AIDS-related cancers (e.g., AIDS-related lymphoma), anal cancer, basal cell carcinoma, bile duct cancer (e.g., extrahepatic), bladder cancer, bone cancer, (osteosarcoma and malignant fibrous histiocytoma), breast cancer, cervical cancer, colon cancer, colorectal cancer, endometrial cancer (e.g., uterine cancer), ependymoma, esophageal cancer, eye cancer (e.g., intraocular melanoma and retinoblastoma), gastric (stomach) cancer, germ cell tumor, (e.g., extracranial, extragonadal, ovarian), head and neck cancer, leukemia, lip and oral cavity cancer, liver cancer, lung cancer (e.g., small cell lung cancer, non-small cell lung cancer, adenocarcinoma of the lung, and squamous carcinoma of the lung), ovarian cancer, pancreatic cancer, pituitary tumor, prostate cancer, renal cancer, skin cancer, small intestine cancer, squamous cell cancer, testicular cancer, throat cancer, thyroid cancer, urethral cancer, and post-transplant lymphoproliferative disorder (PTLD).

In some embodiments, the cancer is a lymphoid cancer (e.g., lymphoma).

In some embodiments, the cancer is a B-cell cancer. In some embodiments, the cancer is precursor B-cell cancers (e.g., precursor B-lymphoblastic leukemia/lymphoma) and peripheral B-cell cancers (e.g., B-cell chronic lymphocytic leukemia/prolymphocytic leukemia/small lymphocytic lymphoma (small lymphocytic (SL) NHL), lymphoplasmacytoid lymphoma/immunocytoma, mantel cell lymphoma, follicle center lymphoma, follicular lymphoma (e.g., cytologic grades: I (small cell), II (mixed small and large cell), III (large cell) and/or subtype: diffuse and predominantly small cell type), low grade/follicular non-Hodgkin's lymphoma (NHL), intermediate grade/follicular NHL, marginal zone B-cell lymphoma (e.g., extranodal (e.g., MALT-type +/− monocytoid B cells) and/or Nodal (e.g., +/− monocytoid B cells)), splenic marginal zone lymphoma (e.g., +/− villous lymphocytes), Hairy cell leukemia, plasmacytoma/plasma cell myeloma (e.g., myeloma and multiple myeloma), diffuse large B-cell lymphoma (e.g., primary mediastinal (thymic) B-cell lymphoma), intermediate grade diffuse NHL, Burkitt's lymphoma, High-grade B-cell lymphoma, Burkitt-like, high grade immunoblastic NHL, high grade lymphoblastic NHL, high grade small non-cleaved cell NHL, bulky disease NHL, AIDS-related lymphoma, and Waldenstrom's macroglobulinemia).

In some embodiments, the cancer is a T-cell and/or putative NK-cell cancer. In some embodiments, the cancer is precursor T-cell cancer (precursor T-lymphoblastic lymphoma/leukemia) and peripheral T-cell and NK-cell cancers (e.g., T-cell chronic lymphocytic leukemia/prolymphocytic leukemia, and large granular lymphocyte leukemia (LGL) (e.g., T-cell type and/or NK-cell type), cutaneous T-cell lymphoma (e.g., mycosis fungoides/Sezary syndrome), primary T-cell lymphomas unspecified (e.g., cytological categories (e.g., medium-sized cell, mixed medium and large cell), large cell, lymphoepitheloid cell, subtype hepatosplenic γδ T-cell lymphoma, and subcutaneous panniculitic T-cell lymphoma), angioimmunoblastic T-cell lymphoma (AILD), angiocentric lymphoma, intestinal T-cell lymphoma (e.g., +/− enteropathy associated), adult T-cell lymphoma/leukemia (ATL), anaplastic large cell lymphoma (ALCL) (e.g., CD30+, T- and null-cell types), anaplastic large-cell lymphoma, and Hodgkin's like).

In some embodiments, the cancer is Hodgkin's disease.

In some embodiments, the cancer is leukemia. In some embodiments, the cancer is chronic myelocytic I (granulocytic) leukemia, chronic myelogenous, and chronic lymphocytic leukemia (CLL), acute lymphoblastic leukemia (ALL), acute myeloid leukemia, acute lymphocytic leukemia, and acute myelocytic leukemia (e.g., myeloblastic, promyelocytic, myelomonocytic, monocytic, and erythroleukemia).

In some embodiments, the cancer is a liquid tumor or plasmacytoma. In some embodiments, the cancer is extramedullary plasmacytoma, a solitary myeloma, and multiple myeloma. In some embodiments, the plasmacytoma is multiple myeloma.

In some embodiments, the cancer is lung cancer. In some embodiments, the RNAi molecule targets all or a portion of the PI3K gene (including genetic mutations thereof and polymorphisms thereof).

In some embodiments, the cancer is colon cancer. In some embodiments, the RNAi molecule targets all or a portion of a gene selected from: the MSH2 gene (including genetic mutations thereof and polymorphisms thereof), the MLH1 gene (including genetic mutations thereof and polymorphisms thereof), the PMS2 gene (including genetic mutations thereof and polymorphisms thereof), the MSH6 gene (including genetic mutations thereof and polymorphisms thereof), the PMS 1 gene (including genetic mutations thereof and polymorphisms thereof), the APC gene (including genetic mutations thereof and polymorphisms thereof), or a combination thereof.

In some embodiments, the cancer is prostate cancer. In some embodiments, the RNAi molecule targets all or a portion of the following genes (including mutations and polymorphisms thereof): prostate-cancer-gene-3 (PCA3), HPC1, PCAP, CAPB, HPC2, HPC20, HPCX, MSR1, ELAC2, or a combination thereof. In some embodiments, the prostate cancer is an adenocarcinoma. In some embodiments, the prostate cancer is a sarcoma, neuroendocrine tumor, small cell cancer, ductal cancer, or a lymphoma. In some embodiments, the prostate cancer is stage A prostate cancer (the cancer cannot be felt during a rectal exam). In some embodiments, the prostate cancer is stage B prostate cancer (i.e., the tumor involves more tissue within the prostate, it can be felt during a rectal exam, or it is found with a biopsy that is done because of a high PSA level). In some embodiments, the prostate cancer is stage C prostate cancer (i.e., the cancer has spread outside the prostate to nearby tissues). In some embodiments, the prostate cancer is stage D prostate cancer. In some embodiments, the prostate cancer is androgen independent prostate cancer (AIPC). In some embodiments, the prostate cancer is androgen dependent prostate cancer. In some embodiments, the prostate cancer is refractory to hormone therapy. In some embodiments, the prostate cancer is substantially refractory to hormone therapy. In some embodiments, the prostate cancer is refractory to chemotherapy. In some embodiments, the prostate cancer is metastatic prostate cancer. In some embodiments, the individual is a human who has a gene, genetic mutation, or polymorphism associated with prostate cancer (e.g., RNASEL/HPC1, ELAC2/HPC2, SR-A/MSR1, CHEK2, BRCA2, PON1, OGG1, MIC-1, TLR4, and PTEN) or has one or more extra copies of a gene associated with prostate cancer. In some embodiments, the prostate cancer is HER2 positive. In some embodiments, the prostate cancer is HER2 negative.

In some embodiments, the cancer has metastasized and is characterized by circulating tumor cells.

### Breast Cancer

In some embodiments, the cancer is breast cancer. In some embodiments, the breast cancer is mammary ductal carcinoma. In some embodiments, the breast cancer is Stage 0 (i.e., pre-malignant). In some embodiments, the breast cancer is Stage 1-3. In some embodiments, the breast cancer is Stage 4 (i.e., advanced and/or metastatic). In some embodiments, the breast cancer is in situ. In some embodiments, the breast cancer is invasive. In some embodiments, the tumor cells of the breast cancer are well differentiated (low grade), moderately differentiated (intermediate grade), or poorly differentiated (high grade). In some embodiments, the breast cancer is ER+. In some embodiments, the breast cancer is HER2+. In some embodiments, the breast cancer is basal-like or triple negative.

Breast cancer genes that are known to be vulnerable to hypermethylation and subsequent degrees of gene transcription and expression silencing include, e.g. cyclin D2, RARbeta2, twist, BRCA1, maspin, estrogen receptor, progesterone receptor, and e-cadherin. Other genes having promoters that can be methylated but that are not necessarily present in a breast context include e.g. p16 (INK4a), P 15 (INK4b), P 14 (ARF), death associated protein (DAP), retinoblastoma Rb, and von-Hippel-Lindaur (VHL) gene. In some embodiments, the RNAi molecule (e.g., siRNA molecules, miRNA molecules, and analogues thereof) targets the region of a promoter comprising a CpG island of any of the aforementioned genes. The composition may comprise one or more or all or several of these classes of agents that relate to and/or affect methylation or demethylation at CpG sites on promoters for breast cancer-related genes. Antagonists or inhibitors can be any molecule capable of antagonizing or inhibiting the target bio-activity. Thus, for example, antagonists or inhibitors can be for example small organic molecules, proteins, polypeptides, peptides, oligonucleotides, lipids, carbohydrates, polymers and the like.

In some embodiments, the RNAi molecule targets all or a portion of the following genes (including mutations and polymorphisms thereof): BRCA1, BRCA2, CDH1, PTEN, STK11, TP53, AR, ATM, BARD1, BRIP1, CHEK2, DIRAS3, ERBB2, NBN, PALB2, RAD50, RAD51, or combinations thereof.

In some embodiments, an RNAi molecule (e.g., siRNA molecules, miRNA molecules, and analogues thereof) disclosed herein is delivered intraductally to a breast duct in a patient. Preferably the duct has been previously identified as having premalignant (e.g. hyperplastic and/or atypical) or malignant (carcinoma) cells and thus been identified as a target for the local treatment protocol proposed in the method. The delivery to the duct can be accomplished by accessing a breast duct with a delivery tool (e.g. a catheter, cannula, or the like) and infusing the agent (in a suitable medium or solution for delivery of the active agent) into the duct to contact target ductal epithelial cells lining the duct. The delivery can also be accomplished e.g. by pump delivery, time-release capsule placed in the duct, and the like.

### Administration and Formulations

Disclosed herein, in certain embodiments, is a formulation, comprising: (a) an RNAi molecule comprising at least one of a locked nucleic acid (LNA), an unlocked nucleic acid (UNA), a bridged nucleic acid (BNA), a glycerol nucleic acid (GNA), or a combination thereof; and (b) an RNAi carrier. In some embodiments, the carrier provides for one or more of the following: stability for shortened duplexes, reduction or prevention of sense strand loading, reduction or prevention of seed region microRNA adverse side effects and reduction of non-specific immunoactivation. In some embodiments, the RNAi carrier is a di-lipid amino acid (DILA²). In some embodiments, the RNAi carrier is a Krebs Cycle analog. In some embodiments, the RNAi carrier is a Krebs Cycle analog and wherein the Krebs Cycle analog reduces or prevents cytotoxicity. In some embodiments, the nucleic acid is a double stranded RNA.

Disclosed herein, in certain embodiments, is a formulation, comprising: (a) an RNA or an RNA analog; and (b) a Krebs Cycle analog RNAi carrier. In some embodiments, the RNA or RNA analog comprises a locked nucleic acid (LNA), an unlocked nucleic acid (UNA), a bridged nucleic acid (BNA), a glycerol nucleic acid (GNA), or a combination thereof.

Disclosed herein, in certain embodiments, is a formulation, comprising: (a) locked nucleic acid (LNA), an unlocked nucleic acid (UNA), a bridged nucleic acid (BNA) oligomer, a glycerol nucleic acid (GNA) analog, or a combination thereof; and (b) a Krebs Cycle analog RNAi carrier.

Disclosed herein, in certain embodiments, is a formulation, comprising: (a) an RNAi molecule comprising at least one glycerol nucleic acid (GNA); and (b) a Krebs Cycle analog RNAi carrier.

Nucleic acid molecules disclosed herein are administered to an individual in need thereof by any suitable method. In some embodiments, nucleic acid molecules disclosed herein are administered to an individual in need thereof by encapsulation in liposomes, by iontophoresis, or by incorporation into other vehicles, such as hydrogels, cyclodextrins, biodegradable nanocapsules, and bioadhesive microspheres, or by proteinaceous vectors. Alternatively, nucleic acid molecules disclosed herein are locally delivered by direct injection or by use of an infusion pump.

In some embodiments, nucleic acid molecules disclosed herein are administered before, during, or immediately after tumor surgery. In another embodiment, a formulation is administered by intravenous injection. In some embodiments, nucleic acid molecules disclosed herein are administered locally at the site of a surgery. Injection of nucleic acid molecules disclosed herein, whether intravenous, subcutaneous, intramuscular, or intradermal, is by any suitable method. In some embodiments, nucleic acid molecules disclosed herein are administered using standard needle and syringe methodologies, or by needle-free technologies.

RNAi molecules are administered in any suitable formulation. In some embodiments, a formulation comprises any suitable excipient. In some embodiments, a formulation comprises a pharmaceutically acceptable carrier, diluent, excipient, adjuvant, emulsifier, buffer, stabilizer, preservative, and the like.

### Carriers

In some embodiments, a formulation comprising an RNAi molecule (e.g., siRNA molecules, miRNA molecules, and analogues thereof) comprises a carrier.

In some embodiments, the carrier is a liposome. In some embodiments, the liposome is a surface modified liposome. In some embodiments, the liposome comprises poly(ethylene glycol) lipids (PEG-modified, or long-circulating liposomes or stealth liposomes).

In some embodiments, the carrier is a di-lipid amino acid (DILA²). For disclosures re DILA² see U.S. patent application Ser. No. 12/114,284.

In some embodiments, the RNAi carrier is a Krebs Cycle analog. In some embodiments, the Krebs Cycle analog reduces or prevents cytotoxicity.

This invention provides a range of Krebs Cycle analogs which are lipophilic compounds for use in delivery and administration of RNAi molecules. The Krebs Cycle analogs of this disclosure are molecules containing Krebs Cycle derivative (e.g., citrate, isocitrate, α-ketoglutarate, succinyl-CoA, succinate, fumarate, malate, oxaloacetate) and one or more lipophilic tails.

In some embodiments, the Krebs Cycle analogs provide relatively low cytotoxicity, and correspondingly, a cytoprotective effect relative to certain other lipids. In some embodiments, the Krebs Cycle analogs are pharmaceutically-acceptable, biodegradable, or biocompatible.

Krebs Cycle analogs can be cationic or non-cationic, where non-cationic includes neutral and anionic. As used herein, the physical state of a species refers to an environment having pH about 7, unless otherwise specified.

Krebs Cycle analogs of this disclosure may exhibit low cytotoxicity. In some embodiments, Krebs Cycle analogs of this disclosure may provide cytoprotective effects relative to lipids of other structures.

In some aspects, Krebs Cycle analogs of this disclosure may provide delivery of an RNAi molecule in a releasable form. Releasable forms and compositions are designed to provide sufficient uptake of an agent by a cell to provide a therapeutic effect.

Releasable forms include Krebs Cycle analogs that bind and release an RNAi molecule. In some embodiments, release of the active agent may be provided by an acid-labile linker.

Examples of acid-labile linkers include linkers containing an orthoester group, a hydrazone, a cis-acetonyl, an acetal, a ketal, a silyl ether, a silazane, an imine, a citraconic anhydride, a maleic anhydride, a crown ether, an azacrown ether, a thiacrown ether, a dithiobenzyl group, a cis-aconitic acid, a cis-carboxylic alkatriene, methacrylic acid, and mixtures thereof.

Examples of acid-labile groups and linkers are given in U.S. Pat. Nos. 7,098,032; 6,897,196; 6,426,086; 7,138,382; 5,563,250; and 5,505,931, all of which are incorporated by reference for such disclosures.

Releasable forms of Krebs Cycle analogs of this disclosure include molecules that bind an active agent and discharge a moiety that assists in release of the agent. In some embodiments, a Krebs Cycle analog may include a group which releases a small molecule such as ethanol that assists in delivering an agent to a cell. A Krebs Cycle analog may bind an active agent and, subsequent to contact with a cell, or subsequent to transport within a biological compartment having a local pH lower than physiological pH, be hydrolyzed in an acidic environment to release ethanol to assist in delivery of the agent. In some embodiments, a small molecule such as ethanol, which assists in delivery of the agent, may be bound to a lipid component.

In some embodiments, a Krebs Cycle analog may be admixed with a compound that releases a small molecule such as ethanol to assists in delivering an agent to a cell.

Releasable forms of Krebs Cycle analogs of this disclosure include Krebs Cycle analogs which may bind an RNAi molecule and, subsequent to contact with a cell, or subsequent to transport within a biological compartment having a local pH lower than physiological pH, be modulated in an acidic environment into a cationic form to assist in release of the RNAi molecule.

In some embodiments, a Krebs Cycle analog may bind an RNAi molecule, and may be admixed with a compound that can be modulated in an acidic environment into a cationic form to assist in release of the RNAi molecule.

Examples of hydrolysable and modulatable groups are given in U.S. Pat. Nos. 6,849,272; 6,200,599; as well as Z. H. Huang and F. C. Szoka, “Bioresponsive liposomes and their use for macromolecular delivery,” in: G. Gregoriadis (ed.), Liposome Technology, 3rd ed. (CRC Press 2006), all of which are incorporated by reference for such disclosures.

In some embodiments, releasable forms of Krebs Cycle analogs of this disclosure include Krebs Cycle analogs which can bind an RNAi molecule, and may be admixed with a lipid or compound that can be modulated in an acidic environment into a neutral form to assist in release of the RNAi molecule. The acidic environment may be entered subsequent to contact with a cell, or subsequent to transport within a biological compartment having a local pH lower than physiological pH.

Examples of lipids which are modulatable from anionic to neutral forms include cholesteryl hemisuccinate (CHEMS) as described in U.S. Pat. Nos. 6,897,196; 6,426,086; and 7,108,863, all of which are incorporated by reference for such disclosures.

In some embodiments, releasable forms of Krebs Cycle analogs of this disclosure include Krebs Cycle analogs which can bind an active agent, and may be admixed with a pH-sensitive polymeric material.

Examples of pH-sensitive polymeric materials are given in U.S. Pat. No. 6,835,393, which is incorporated by reference for such disclosures.

In some embodiments, release of the RNAi molecule may be provided by an enzyme-cleavable peptide.

In some embodiments, the Krebs Cycle analog comprises a Krebs Cycle derivative (e.g., citrate, isocitrate, α-ketoglutarate, succinyl-CoA, succinate, fumarate, malate, oxaloacetate) wherein each of the terminal carboxylic acid groups of the Krebs Cycle derivative are functionalized to provide a lipophilic tail comprising (a) a naturally-occurring or synthetic lipid, phospholipid, glycolipid, triacylglycerol, glycerophospholipid, sphingolipid, ceramide, sphingomyelin, cerebroside, or ganglioside; (b) a substituted or unsubstituted C(3-22)alkyl, C(6-12)cycloalkyl, C(6-12)cycloalkyl-C(3-22)alkyl, C(3-22)alkenyl, C(3-22)alkynyl, C(3-22)alkoxy, or C(6-12)alkoxy-C(3-22)alkyl; or (c) a lipophilic tail of any other naturally-occurring or synthetic lipid, or a lipophilic tail of any one of the delivery lipids described in U.S. patent application Ser. No. 12/114,284; and the intervening carbon atoms between the two terminal carboxylic acid groups include a guanidine, alkylguanidine, dialkylguanidine, amidine, alkylamidine, or dialkylamidine containing side chain. Details regarding the synthesis of such carriers can be found in U.S. patent application Ser. No. 12/114,284, which is herein incorporated by reference for such disclosures.

The term “alkyl” as used herein refers to a saturated or unsaturated, branched or unbranched, substituted or unsubstituted aliphatic group containing from 1-22 carbon atoms. This definition applies to the alkyl portion of other groups such as, for example, alkoxy, alkanoyl, aralkyl, and other groups defined below. As used herein, the term “C(1-5)alkyl,” for example, includes C(1)alkyl, C(2)alkyl, C(3)alkyl, C(4)alkyl, and C(5)alkyl. Likewise, the term “C(1-22)alkyl,” for example, includes C(1)alkyl, C(2)alkyl, C(3)alkyl, C(4)alkyl, C(5)alkyl, C(6)alkyl, C(7)alkyl, C(8)alkyl, C(9)alkyl, C(10)alkyl, C(11)alkyl, C(12)alkyl, C(13)alkyl, C(14)alkyl, C(15)alkyl, C(16)alkyl, C(17)alkyl, C(18)alkyl, C(19)alkyl, C(20)alkyl, C(21)alkyl, and C(22)alkyl.

A pharmaceutically acceptable salt of a carrier of this invention which is sufficiently basic may be an acid-addition salt with, for example, an inorganic or organic acid such as hydrochloric, hydrobromic, sulfuric, nitric, phosphoric, chlorosulfonic, trifluoroacetic, citric, maleic, acetic, propionic, oxalic, malic, maleic, malonic, fumaric, or tartaric acids, and alkane- or arenesulfonic acids such as methanesulfonic, ethanesulfonic, benzenesulfonic, chlorobenzenesulfonic, toluenesulfonic, naphthalenesulfonic, naphthalenedisulfonic, and camphorsulfonic acids.

In some embodiments, the lipophilic tails impart sufficient lipophilic character or lipophilicity, such as defined by water/octanol partitioning, to provide delivery across a membrane or uptake by a cell. These tails provide, when used in an amino acid lipid structure, an amphipathic molecule. Lipid-like tails may be derived from phospholipids, glycolipids, triacylglycerols, glycerophospholipids, sphingolipids, ceramides, sphingomyelins, cerebrosides, or gangliosides, among others, and may contain a steroid.

In certain embodiments, each or both lipid-like tails has a glycerol backbone.

In some embodiments, each lipophilic tail is independently a C3alkyl, C4alkyl, C5alkyl, C6alkyl, C7alkyl, C8alkyl, C9alkyl, C10alkyl, C11alkyl, C12alkyl, C13alkyl, C14alkyl, C15alkyl, C16alkyl, C17alkyl, C18alkyl, C19alkyl, C20alkyl, C21alkyl, or C22alkyl.

In some embodiments, each lipophilic tail is independently selected from lipophilic tails having one of the following structures:

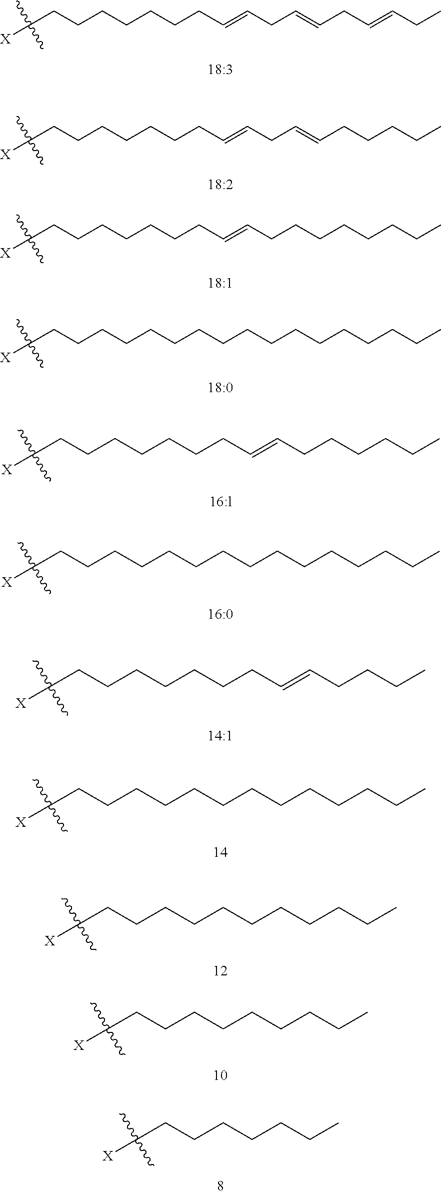

In the structures above, X represents the atom of the tail that is directly attached to Krebs Cycle derivative (e.g., citrate, isocitrate, α-ketoglutarate, succinyl-CoA, succinate, fumarate, malate, oxaloacetate) residue terminus, and is counted as one of the atoms in the numerical designation, for example, “18:3.” In some embodiments, X may be a carbon, nitrogen, or oxygen atom.

In some embodiments, each lipophilic tail is independently selected from lipophilic tails having one of the following structures:

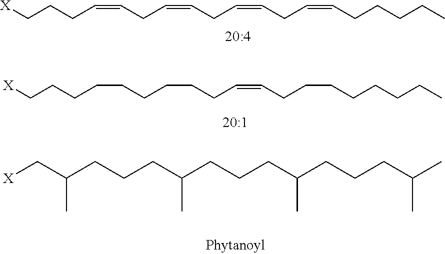

where X is as defined above.

In some embodiments, each lipophilic tail independently comprises a cholesterol, a sterol, or a steroid such as gonanes, estranes, androstanes, pregnanes, cholanes, cholestanes, ergostanes, campestanes, poriferastanes, stigmastanes, gorgostanes, lanostanes, cycloartanes, as well as sterol or zoosterol derivatives of any of the foregoing, and their biological intermediates and precursors, which may include, for example, cholesterol, lanosterol, stigmastanol, dihydrolanosterol, zymosterol, zymostenol, desmosterol, 7-dehydrocholesterol, and mixtures and derivatives thereof.

In some embodiments, each lipophilic tail independently comprises fatty acid-like tails such as tails from myristic acid (C14:0)alkenyl, palmitic acid (C16:0)alkenyl, stearic acid (C18:0)alkenyl, oleic acid (C18:1, double bond at carbon 9)alkenyl, linoleic acid (C18:2, double bond at carbon 9 or 12)alkenyl, linonenic acid (C18:3, double bond at carbon 9, 12, or 15)alkenyl, arachidonic acid (C20:4, double bond at carbon 5, 8, 11, or 14)alkenyl, and eicosapentaenoic acid (C20:5, double bond at carbon 5, 8, 11, 14, or 17)alkenyl.

In some embodiments, each lipophilic tail comprises an isoprenoid.

A pharmaceutically acceptable salt of a carrier disclosed herein which is sufficiently acidic may be an alkali metal salt, for example, a sodium or potassium salt, or an alkaline earth metal salt, for example, a calcium or magnesium salt, or a zinc or manganese salt, or an ammonium salt or a salt with an organic base which provides a physiologically-acceptable cation, for example, a salt with methylamine, dimethylamine, trimethylamine, triethylamine, ethanolamine, diethanolamine, triethanolamine, ethylenediamine, tromethamine, N-methylglucamine, piperidine, morpholine or tris-(2-hydroxyethyl)amine, and including salts of amino acids such as arginate, and salts of organic acids such as glucuronic or galactunoric acids.

A salt or pharmaceutically-acceptable salt of a carrier disclosed herein which contains an RNAi molecule and a lipid, peptide, or protein, among other components, may contain a salt complex of the interfering-RNA agent and the lipid, peptide, or protein. A salt complex of the RNAi molecule and the lipid, peptide, or protein may be formed from a pharmaceutically-acceptable salt of an RNAi molecule, or from a pharmaceutically-acceptable salt of the lipid, peptide, or protein.

A carrier disclosed herein may contain both basic and acidic functionalities that may allow the compounds to be made into either a base or acid addition salt.

A carrier disclosed herein may have one or more chiral centers and/or geometric isomeric centers (E- and Z-isomers), and it is to be understood that the invention encompasses all such optical isomers, diastereoisomers, geometric isomers, and mixtures thereof.

This invention encompasses any and all tautomeric, solvated or unsolvated, hydrated or unhydrated forms, as well as any atom isotope forms of the carriers disclosed herein.

In some embodiments, the use of a carrier potentially localizes the RNAi molecule, for example, in certain tissue types, such as the tissues of the reticular endothelial system (RES). In some embodiments, a liposome formulation facilitates the association of drug with the surface of specific cells.

### Formulations

Disclosed herein, in some embodiments, is a pharmaceutical formulation comprising an RNAi molecule. Pharmaceutical comprise one or more physiologically acceptable carriers such as di-lipid amino acid (DILA2) and/or a Krebs Cycle analog. Proper formulation is dependent upon the route of administration chosen. A summary of pharmaceutical formulations is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins, 1999).

In some embodiments, a pharmaceutical formulation, comprising an RNAi molecule (e.g., siRNA molecules, miRNA molecules, and analogues thereof) further comprises pharmaceutically acceptable excipient(s) such as preserving, stabilizing, wetting or emulsifying agents, solution promoters, salts for regulating the osmotic pressure, and/or buffers.

The pharmaceutical formulations described herein are optionally administered to an individual by multiple administration routes, including but not limited to, oral, parenteral (e.g., intravenous, subcutaneous, intramuscular), intranasal, buccal, topical, rectal, or transdermal administration routes. The pharmaceutical formulations described herein include, but are not limited to, aqueous liquid dispersions, self-emulsifying dispersions, solid solutions, liposomal dispersions, aerosols, solid dosage forms, powders, immediate release formulations, controlled release formulations, fast melt formulations, tablets, capsules, pills, delayed release formulations, extended release formulations, pulsatile release formulations, multiparticulate formulations, and mixed immediate and controlled release formulations.

The pharmaceutical formulations described herein are administered via any suitable dosage form, including but not limited to, aqueous oral dispersions, liquids, gels, syrups, elixirs, slurries, suspensions and the like, for oral ingestion by an individual to be treated, solid oral dosage forms, aerosols, controlled release formulations, fast melt formulations, effervescent formulations, lyophilized formulations, tablets, powders, pills, dragees, capsules, modified release formulations, delayed release formulations, extended release formulations, pulsatile release formulations, multiparticulate formulations, and mixed immediate release and controlled release formulations.

In some embodiments, a formulation disclosed herein is formulated for parenteral injection (e.g., via injection or infusion, including intraarterial, intracardiac, intradermal, intraduodenal, intramedullary, intramuscular, intraosseous, intraperitoneal, intrathecal, intravascular, intravenous, intravitreal, epidural and subcutaneous). In some embodiments, a formulation disclosed herein is administered as a sterile solution, suspension or emulsion.

In some embodiments, a formulation for parenteral administration includes aqueous and non-aqueous (oily) sterile injection solutions of the active compounds which may contain antioxidants, buffers, bacteriostats and solutes which render the formulation isotonic with the blood of the intended recipient; and aqueous and non-aqueous sterile suspensions which may include suspending agents and thickening agents. In some embodiments, a formulation for parenteral administration includes suitable stabilizers or agents which increase the solubility of the compounds to allow for the preparation of highly concentrated solutions.

In some embodiments, a compound disclosed herein is administered as an aqueous suspension. In some embodiments, an aqueous suspension comprises water, Ringer's solution or isotonic sodium chloride solution.

In some embodiments, a formulation formulated for parenteral administration is administered as a single bolus shot. In some embodiments, a formulation formulated for parenteral administration is administered via a continuous intravenous delivery device (e.g., Deltec CADD-PLUS™ model 5400 intravenous pump).

In some embodiments, a formulation for injection is presented in unit dosage form, e.g., in ampoules or in multi-dose containers, with an added preservative. In some embodiments, a formulation for injection is stored in powder form or in a freeze-dried (lyophilized) condition requiring only the addition of the sterile liquid carrier, for example, saline or sterile pyrogen-free water, immediately prior to use.

In some embodiments, a formulation disclosed herein is administered by depot preparation. In some embodiments, a depot preparation is administered by implantation (for example subcutaneously or intramuscularly) or by intramuscular injection.

Transdermal formulations described herein include at least three components: (1) an agent; (2) a penetration enhancer; and (3) an aqueous adjuvant. In addition, transdermal formulations include components such as, but not limited to, gelling agents, creams and ointment bases, and the like. In some embodiments, the transdermal formulation further includes a woven or non-woven backing material to enhance absorption and prevent the removal of the transdermal formulation from the skin. In other embodiments, the transdermal formulations described herein maintain a saturated or supersaturated state to promote diffusion into the skin.

Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents are optionally present.

For administration by inhalation, the pharmaceutical formulations disclosed herein are optionally in a form of an aerosol, a mist or a powder. Pharmaceutical formulations described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol, the dosage unit is determined by providing a valve to deliver a metered amount. Capsules and cartridges of, such as, by way of example only, gelatin for use in an inhaler or insufflator are formulated containing a powder mix and a suitable powder base such as lactose or starch.

Buccal dosage forms described herein optionally further include a bioerodible (hydrolysable) polymeric carrier that also serves to adhere the dosage form to the buccal mucosa. The buccal dosage form is fabricated so as to erode gradually over a predetermined time period. Buccal drug delivery avoids the disadvantages encountered with oral drug administration, e.g., slow absorption, degradation of the agent by fluids present in the gastrointestinal tract and/or first-pass inactivation in the liver. The bioerodible (hydrolysable) polymeric carrier generally comprises hydrophilic (water-soluble and water-swellable) polymers that adhere to the wet surface of the buccal mucosa. Examples of polymeric carriers useful herein include acrylic acid polymers and co, e.g., those known as “carbomers” (Carbopol®, which is obtained from B.F. Goodrich, is one such polymer). Other components also be incorporated into the buccal dosage forms described herein include, but are not limited to, disintegrants, diluents, binders, lubricants, flavoring, colorants, preservatives, and the like. For buccal or sublingual administration, the formulations optionally take the form of tablets, lozenges, or gels formulated in a conventional manner.

In some embodiments, formulations suitable for transdermal administration employ transdermal delivery devices and transdermal delivery patches and are lipophilic emulsions or buffered, aqueous solutions, dissolved and/or dispersed in a polymer or an adhesive. Such patches are optionally constructed for continuous, pulsatile, or on demand delivery of pharmaceutical agents. Still further, transdermal delivery is optionally accomplished by means of iontophoretic patches and the like. Additionally, transdermal patches provide controlled delivery. The rate of absorption is optionally slowed by using rate-controlling membranes or by trapping an agent within a polymer matrix or gel. Conversely, absorption enhancers are used to increase absorption. An absorption enhancer or carrier includes absorbable pharmaceutically acceptable solvents to assist passage through the skin. For example, transdermal devices are in the form of a bandage comprising a backing member, a reservoir containing an agent optionally with carriers, optionally a rate controlling barrier to deliver a an agent to the skin of the host at a controlled and predetermined rate over a prolonged period of time, and means to secure the device to the skin.

In some embodiments, An RNAi molecule disclosed herein is administered topically and formulated into a variety of topically administrable formulations, such as solutions, suspensions, lotions, gels, pastes, medicated sticks, balms, creams or ointments. Such pharmaceutical formulations optionally contain solubilizers, stabilizers, tonicity enhancing agents, buffers and preservatives.

An RNAi molecule disclosed herein is also optionally formulated in rectal formulations such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the formulations, a low-melting wax such as, but not limited to, a mixture of fatty acid glycerides, optionally in combination with cocoa butter is first melted.

In some embodiments, an RNAi molecule (e.g., siRNA molecules, miRNA molecules, and analogues thereof) is administered in a controlled release formulation. In general, controlled release drug formulations impart control over the release of drug with respect to site of release and time of release within the body. Controlled release refers to immediate release, delayed release, extended release and pulsatile release. Many advantages are offered by controlled release. First, controlled release of a pharmaceutical agent allows less frequent dosing and thus minimizes repeated treatment. Second, controlled release treatment results in more efficient drug utilization and less of the compound remaining as a residue. Third, controlled release offers the possibility of localized drug delivery by placement of a delivery device or formulation at the site of disease. Fourth, controlled release offers the opportunity to administer and release two or more different drugs, each having a unique release profile, or to release the same drug at different rates or for different durations, by means of a single dosage unit.

In some embodiments, an RNAi molecule (e.g., siRNA molecules, miRNA molecules, and analogues thereof) is incorporated within controlled release particles, lipid complexes, liposomes, nanoparticles, microspheres, microparticles, nanocapsules or other agents which enhance or facilitate the localized delivery of RNAi molecule. In some embodiments, a single enhanced viscosity formulation is used, while in other embodiments, a pharmaceutical formulation that comprises a mixture of two or more distinct enhanced viscosity formulations is used. In some embodiments, combinations of sols, gels and/or biocompatible matrices are also employed to provide desirable characteristics of the controlled release formulations or formulations. In certain embodiments, the controlled release formulations or formulations are cross-linked by one or more agents to alter or improve the properties of the formulation.

Examples of microspheres relevant to the pharmaceutical formulations disclosed herein include: Luzzi, L. A., J. Pharm. Psy. 59:1367 (1970); U.S. Pat. No. 4,530,840; Lewis, D. H., “Controlled Release of Bioactive Agents from Lactides/Glycolide Polymers” in Biodegradable Polymers as Drug Delivery Systems, Chasin, M. and Langer, R., eds., Marcel Decker (1990); U.S. Pat. No. 4,675,189; Beck et al., “Poly(lactic acid) and Poly(lactic acid-co-glycolic acid) Contraceptive Delivery Systems,” in Long Acting Steroid Contraception, Mishell, D. R., ed., Raven Press (1983); U.S. Pat. No. 4,758,435; U.S. Pat. No. 3,773,919; U.S. Pat. No. 4,474,572. Examples of protein therapeutics formulated as microspheres include: U.S. Pat. No. 6,458,387; U.S. Pat. No. 6,268,053; U.S. Pat. No. 6,090,925; U.S. Pat. No. 5,981,719; and U.S. Pat. No. 5,578,709, and are herein incorporated by reference for such disclosure.

Microspheres usually have a spherical shape, although irregularly-shaped microparticles are possible. Microspheres may vary in size, ranging from submicron to 1,000 micron diameters. Microspheres suitable for use with RNAi formulations disclosed herein are submicron to 250 micron diameter microspheres, allowing administration by injection with a standard gauge needle. The microspheres can thus be prepared by any method which produces microspheres in a size range acceptable for use in an injectable formulation. Injection is optionally accomplished with standard gauge needles used for administering liquid formulations.

Suitable examples of polymeric matrix materials for use in the controlled release particles herein include poly(glycolic acid), poly-d,l-lactic acid, poly-l-lactic acid, copolymers of the foregoing, poly(aliphatic carboxylic acids), copolyoxalates, polycaprolactone, polydioxonene, poly(orthocarbonates), poly(acetals), poly(lactic acid-caprolactone), polyorthoesters, poly(glycolic acid-caprolactone), polydioxonene, polyanhydrides, polyphosphazines, and natural polymers including albumin, casein, and some waxes, such as, glycerol mono- and distearate, and the like. Various commercially available poly(lactide-co-glycolide) materials (PLGA) are optionally used in the method disclosed herein. For example, poly(d,l-lactic-co-glycolic acid) is commercially available from Boehringer-Ingelheim as RESOMER RG 503 H. This product has a mole percent formulation of 50% lactide and 50% glycolide. These copolymers are available in a wide range of molecular weights and ratios of lactic acid to glycolic acid. One embodiment includes the use of the polymer poly(d,l-lactide-co-glycolide). The molar ratio of lactide to glycolide in such a copolymer includes the range of from about 95:5 to about 50:50. In other embodiments, PLGA copolymers with polyethylene glycol (PEG) are suitable polymeric matrices for the formulations disclosed herein. For example, PEG-PLGA-PEG block polymers are biodegradable matrices for gel formation that provide high mechanical stability of the resulting gel. Mechanical stabilities of gels using PEG-PLGA-PEG block polymers have been maintained for more than one month in vitro. In some embodiments, PEG-PLGA-PEG block polymers are used to control the release rate of RNAi molecules and/or additional active agents with different physical properties. Particularly, in some embodiments, hydrophilic agents are released more quickly, e.g., approximately 50% of drug release after 24 hours, the remainder released over approximately 5 days, whereas hydrophobic agents are released more slowly, e.g., approximately 80% after 8 weeks.

The molecular weight of the polymeric matrix material is of some importance. The molecular weight should be high enough so that it forms satisfactory polymer coatings, i.e., the polymer should be a good film former. Usually, a satisfactory molecular weight is in the range of 5,000 to 500,000 daltons. The molecular weight of a polymer is also important from the point of view that molecular weight influences the biodegradation rate of the polymer. For a diffusional mechanism of drug release, the polymer should remain intact until all of the drug is released from the microparticles and then degrade. The drug can also be released from the microparticles as the polymeric excipient bioerodes. By an appropriate selection of polymeric materials a microsphere formulation is optionally made such that the resulting microspheres exhibit both diffusional release and biodegradation release properties. This is useful in affording multiphasic release patterns.

A variety of methods are known by which compounds are encapsulated in microspheres. In these methods, RNAi molecules are generally dispersed or emulsified, using stirrers, agitators, or other dynamic mixing techniques, in a solvent containing a wall-forming material. Solvent is then removed from the microspheres, and thereafter the microsphere product is obtained.

In one embodiment, controlled release formulations are made through the incorporation of RNAi molecules into ethylene-vinyl acetate copolymer matrices. (See U.S. Pat. No. 6,083,534, incorporated herein for such disclosure). In another embodiment, RNAi molecules are incorporated into poly(lactic-glycolic acid) or poly-L-lactic acid microspheres. Id. In yet another embodiment, the RNAi moleculesRNAi molecules are encapsulated into alginate microspheres. (See U.S. Pat. No. 6,036,978, incorporated herein for such disclosure). Biocompatible methacrylate-based polymers to encapsulate the formulations are optionally used in the formulations and methods disclosed herein. A wide range of methacrylate-based polymer systems are commerically available, such as the EUDRAGIT polymers marketed by Evonik. One useful aspect of methacrylate polymers is that the properties of the formulation is optionally varied by incorporating various copolymers. For example, poly(acrylic acid-co-methylmethacrylate) microparticles exhibit enhanced mucoadhesion properties as the carboxylic acid groups in the poly(acrylic acid) can form hydrogen bonds with mucin (Park et al., Pharm. Res. (1987) 4(6):457-464). Variation of the ratio between acrylic acid and methylmethacrylate monomers serves to modulate the properties of the co-polymer. Methacrylate-based microparticles have also been used in protein therapeutic formulations (Naha et al., Journal of Microencapsulation 4 Feb., 2008 (online publication)). In one embodiment, the enhanced viscosity formulations described herein comprise microspheres of RNAi molecules wherein the microspheres are formed from a methacrylate polymer or copolymer. In an additional embodiment, the enhanced viscosity formulation described herein comprises microspheres of RNAi molecules wherein the microspheres are mucoadhesive. Other controlled release systems, including incorporation or deposit of polymeric materials or matrices onto solid or hollow spheres containing RNAi molecules are also explicitly contemplated within the embodiments disclosed herein. The types of controlled release systems available without significantly losing activity of the agent are determined using the teachings, examples, and principles disclosed herein

An example of a conventional microencapsulation process for pharmaceutical preparations is shown in U.S. Pat. No. 3,737,337, incorporated herein by reference for such disclosure. The RNAi molecules to be encapsulated or embedded are dissolved or dispersed in the organic solution of the polymer (phase A), using conventional mixers, including (in the preparation of dispersion) vibrators and high-speed stirrers, etc. The dispersion of phase (A), containing the core material in solution or in suspension, is carried out in the aqueous phase (B), again using conventional mixers, such as high-speed mixers, vibration mixers, or even spray nozzles, in which case the particle size of the microspheres will be determined not only by the concentration of phase (A), but also by the emulsate or microsphere size. With conventional techniques for microencapsulation, the microspheres form when the solvent containing an active agent and a polymer is emulsified or dispersed in an immiscible solution by stirring, agitating, vibrating, or some other dynamic mixing technique, often for a relatively long period of time.

Conventional methods for the construction of microspheres are also described in U.S. Pat. No. 4,389,330, and U.S. Pat. No. 4,530,840, incorporated herein by reference for such disclosure. The desired agent is dissolved or dispersed in an appropriate solvent. To the agent-containing medium is added the polymeric matrix material in an amount relative to the active ingredient which gives a product of the desired loading of active agent. Optionally, all of the ingredients of the microsphere product is optionally blended in the solvent medium together. Suitable solvents for the agent and the polymeric matrix material include organic solvents such as acetone, halogenated hydrocarbons such as chloroform, methylene chloride and the like, aromatic hydrocarbon compounds, halogenated aromatic hydrocarbon compounds, cyclic ethers, alcohols, ethyl acetate and the like.

The mixture of ingredients in the solvent is emulsified in a continuous-phase processing medium; the continuous-phase medium being such that a dispersion of microdroplets containing the indicated ingredients is formed in the continuous-phase medium. Naturally, the continuous-phase processing medium and the organic solvent must be immiscible and include water, although nonaqueous media such as xylene and toluene and synthetic oils and natural oils are optionally used. Optionally, a surfactant is added to the continuous-phase processing medium to prevent the microparticles from agglomerating and to control the size of the solvent microdroplets in the emulsion. A preferred surfactant-dispersing medium combination is a 1 to 10 wt. % poly(vinyl alcohol) in water mixture. The dispersion is formed by mechanical agitation of the mixed materials. An emulsion can also be formed by adding small drops of the active agent-wall forming material solution to the continuous phase processing medium. The temperature during the formation of the emulsion is not especially critical but can influence the size and quality of the microspheres and the solubility of the drug in the continuous phase. It is desirable to have as little of the agent in the continuous phase as possible. Moreover, depending on the solvent and continuous-phase processing medium employed, the temperature must not be too low or the solvent and processing medium will solidify or the processing medium will become too viscous for practical purposes, or too high that the processing medium will evaporate, or that the liquid processing medium will not be maintained. Moreover, the temperature of the medium cannot be so high that the stability of the particular agent being incorporated in the microspheres is adversely affected. Accordingly, the dispersion process is optionally conducted at any temperature which maintains stable operating conditions, which preferred temperature being about 15° C. to 60° C., depending upon the drug and excipient selected.

The dispersion which is formed is a stable emulsion and from this dispersion the organic solvent immiscible fluid can optionally be partially removed in the first step of the solvent removal process. The solvent is optionally removed by techniques such as heating, the application of a reduced pressure or a combination of both. The temperature employed to evaporate solvent from the microdroplets is not critical, but should not be that high that it degrades the agent(s) employed in the preparation of a given microparticle, nor should it be so high as to evaporate solvent at such a rapid rate to cause defects in the wall forming material. Generally, from 5 to 75%, of the solvent is removed in the first solvent removal step.

After the first stage, the dispersed microparticles in the solvent immiscible fluid medium are isolated from the fluid medium by any convenient means of separation. Thus, for example, the fluid is optionally decanted from the microsphere or the microsphere suspension filtered. Still other, various combinations of separation techniques are optionally used if desired.

Following the isolation of the microspheres from the continuous-phase processing medium, the remainder of the solvent in the microspheres is removed by extraction. In this step, the microspheres are optionally suspended in the same continuous-phase processing medium used in step one, with or without surfactant, or in another liquid. The extraction medium removes the solvent from the microspheres and yet does not dissolve the microspheres. During the extraction, the extraction medium with dissolved solvent can optionally be removed and replaced with fresh extraction medium. This is best done on a continual basis. Obviously, the rate of extraction medium replenishment of a given process is a variable which can easily be determined at the time the process is performed and, therefore, no precise limits for the rate must be predetermined. After the majority of the solvent has been removed from the microspheres, the microspheres are dried by exposure to air or by other conventional drying techniques such as vacuum drying, drying over a desiccant, or the like. This process is very efficient in encapsulating RNAi molecules since core loadings of up to 80 wt. %, preferably up to 60 wt. % are obtained.

Alternatively, controlled release microspheres containing RNAi molecules are optionally prepared through the use of static mixers. Static or motionless mixers consist of a conduit or tube in which is received a number of static mixing agents. Static mixers provide homogeneous mixing in a relatively short length of conduit, and in a relatively short period of time. With static mixers, the fluid moves through the mixer, rather than some part of the mixer, such as a blade moving through the fluid.

A static mixer is optionally used to create an emulsion. When using a static mixer to form an emulsion, several factors determine emulsion particle size, including the density and viscosity of the various solutions or phases to be mixed, volume ratio of the phases, interfacial tension between the phases, static mixer parameters (conduit diameter; length of mixing element; number of mixing elements), and linear velocity through the static mixer. Temperature is a variable because it affects density, viscosity, and interfacial tension. The controlling variables are linear velocity, sheer rate, and pressure drop per unit length of static mixer.

In order to create microspheres containing RNAi molecules using a static mixer process, an organic phase and an aqueous phase are combined. The organic and aqueous phases are largely or substantially immiscible, with the aqueous phase constituting the continuous phase of the emulsion. The organic phase includes RNAi molecules or as well as a wall-forming polymer or polymeric matrix material. The organic phase is optionally prepared by dissolving RNAi molecules in an organic or other suitable solvent, or by forming a dispersion or an emulsion containing the agent(s). The organic phase and the aqueous phase are pumped so that the two phases flow simultaneously through a static mixer, thereby forming an emulsion which comprises microspheres containing the agent(s) encapsulated in the polymeric matrix material. The organic and aqueous phases are pumped through the static mixer into a large volume of quench liquid to extract or remove the organic solvent. Organic solvent is optionally removed from the microspheres while they are washing or being stirred in the quench liquid. After the microspheres are washed in a quench liquid, they are isolated, as through a sieve, and dried.

In one embodiment, microspheres are prepared using a static mixer is optionally carried out for a variety of techniques used to encapsulate active agents. The process is not limited to the solvent extraction technique discussed above, but can be used with other encapsulation techniques. For example, the process can also be used with a phase separation encapsulation technique. To do so, an organic phase is prepared that comprises RNAi molecules suspended or dispersed in a polymer solution. The non-solvent second phase is free from solvents for the polymer and active agent. A preferred non-solvent second phase is silicone oil. The organic phase and the non-solvent phase are pumped through a static mixer into a non-solvent quench liquid, such as heptane. The semi-solid particles are quenched for complete hardening and washing. The process of microencapsulation includes spray drying, solvent evaporation, a combination of evaporation and extraction, and melt extrusion.

In another embodiment, the microencapsulation process involves the use of a static mixer with a single solvent. This process is described in detail in U.S. application Ser. No. 08/338,805, herein incorporated by reference for such disclosure. An alternative process involves the use of a static mixer with co-solvents. In this process, biodegradable microspheres comprising a biodegradable polymeric binder and RNAi molecules are prepared, which comprises a blend of at least two substantially non-toxic solvents, free of halogenated hydrocarbons to dissolve both the agent and the polymer. The solvent blend containing the dissolved agent and polymer is dispersed in an aqueous solution to form droplets. The resulting emulsion is then added to an aqueous extraction medium preferably containing at least one of the solvents of the blend, whereby the rate of extraction of each solvent is controlled, whereupon the biodegradable microspheres containing the pharmaceutically active agent are formed. This process has the advantage that less extraction medium is required because the solubility of one solvent in water is substantially independent of the other and solvent selection is increased, especially with solvents that are particularly difficult to extract.

Nanoparticles are also contemplated for use with the formulations disclosed herein. Nanoparticles are material structures of about 100 nm or less in size. One use of nanoparticles in pharmaceutical formulations is the formation of suspensions as the interaction of the particle surface with solvent is strong enough to overcome differences in density. Nanoparticle suspensions are optionally sterilized as the nanoparticles are small enough to be subjected to sterilizing filtration (see, e.g., U.S. Pat. No. 6,139,870, herein incorporated by reference for such disclosure). Nanoparticles comprise at least one hydrophobic, water-insoluble and water-indispersible polymer or copolymer emulsified in a solution or aqueous dispersion of surfactants, phospholipids or fatty acids. The RNAi molecules are optionally introduced with the polymer or the copolymer into the nanoparticles.

Lipid nanocapsules are also contemplated herein. Lipid nanocapsules are optionally formed by emulsifying capric and caprylic acid triglycerides (Labrafac W. L. 1349; avg. mw 512), soybean lecithin (LIPOID® S75-3; 69% phosphatidylcholine and other phospholipids), surfactant (for example, SOLUTOL® HS15), a mixture of polyethylene glycol 660 hydroxystearate and free polyethylene glycol 660; NaCl and water. The mixture is stirred at room temperature to obtain an oil emulsion in water. After progressive heating at a rate of 4° C./min under magnetic stirring, a short interval of transparency should occur close to 70° C., and the inverted phase (water droplets in oil) obtained at 85° C. Three cycles of cooling and heating is then applied between 85° C. and 60° C. at the rate of 4° C./min, and a fast dilution in cold water at a temperature close to 0° C. to produce a suspension of nanocapsules. To encapsulate the RNAi moleculesRNAi molecules, the RNAi molecules are optionally added just prior to the dilution with cold water.

The RNAi molecule (e.g., siRNA molecules, miRNA molecules, and analogues thereof) is also inserted into the lipid nanocapsules by incubation for 90 minutes with an aqueous micellar solution. The suspension is then vortexed every 15 minutes, and then quenched in an ice bath for 1 minute.

Suitable surfactants are, by way of example, cholic acid or taurocholic acid salts. Taurocholic acid, the conjugate formed from cholic acid and taurine, is a fully metabolizable sulfonic acid surfactant. An analogue of taurocholic acid, tauroursodeoxycholic acid (TUDCA), is a naturally occurring bile acid and is a conjugate of taurine and ursodeoxycholic acid (UDCA). Other naturally occurring anionic (e.g., galactocerebroside sulfate), neutral (e.g., lactosylceramide) or zwitterionic surfactants (e.g., sphingomyelin, phosphatidyl choline, palmitoyl carnitine) are optionally used to prepare nanoparticles.

The phospholipids are chosen, by way of example, from natural, synthetic or semi-synthetic phospholipids; lecithins (phosphatidylcholine) such as, for example, purified egg or soya lecithins (lecithin E100, lecithin E80 and phospholipons, for example phospholipon 90), phosphatidylethanolamine, phosphatidylserine, phosphatidylinositol, phosphatidylglycerol, dipalmitoylphosphatidylcholine, dipalmitoylglycerophosphatidylcholine, dimyristoylphosphatidylcholine, distearoylphosphatidylcholine and phosphatidic acid or mixtures thereof are used more particularly.

Fatty acids for use with the formulations are chosen from, by way of example, lauric acid, mysristic acid, palmitic acid, stearic acid, isostearic acid, arachidic acid, behenic acid, oleic acid, myristoleic acid, palmitoleic acid, linoleic acid, alpha-linoleic acid, arachidonic acid, eicosapentaenoic acid, erucic acid, docosahexaenoic acid, and the like.

Suitable surfactants can preferably be selected from known organic and inorganic pharmaceutical excipients. Such excipients include various polymers, low molecular weight oligomers, natural products, and surfactants. Preferred surface modifiers include nonionic and ionic surfactants. Two or more surface modifiers are optionally used in combination.

Representative examples of surfactants include cetyl pyridinium chloride, gelatin, casein, lecithin (phosphatides), dextran, glycerol, gum acacia, cholesterol, tragacanth, stearic acid, calcium stearate, glycerol monostearate, cetostearyl alcohol, cetomacrogol emulsifying wax, sorbitan esters, polyoxyethylene alkyl ethers, polyoxyethylene castor oil derivatives, polyoxyethylene sorbitan fatty acid esters; dodecyl trimethyl ammonium bromide, polyoxyethylenestearates, colloidal silicon dioxide, phosphates, sodium dodecylsulfate, carboxymethylcellulose calcium, hydroxypropyl cellulose (HPC, HPC-SL, and HPC-L), hydroxypropyl methylcellulose (HPMC), carboxymethylcellulose sodium, methylcellulose, hydroxyethylcellulose, hydroxypropylcellulose, hydroxypropylmethyl-cellulose phthalate, noncrystalline cellulose, magnesium aluminum silicate, triethanolamine, polyvinyl alcohol (PVA), polyvinylpyrrolidone (PVP), 4-(1,1,3,3-tetaamethylbutyl)-phenol polymer with ethylene oxide and formaldehyde (also known as tyloxapol, superione, and triton), poloxamers, poloxamnines, a charged phospholipid such as dimyristoyl phosphatidyl glycerol, dioctylsulfosuccinate (DOSS); Tetronic 1508, dialkylesters of sodium sulfosuccinic acid, Duponol P, Tritons X-200, Crodestas F-110, p-isononylphenoxypoly-(glycidol), Crodestas SL-40® (Croda, Inc.); and SA9OHCO, which is C₁₈H₃₇CH₂(CON(CH₃)—CH₂(CHOH)₄(CH₂OH)₂ (Eastman Kodak Co.); decanoyl-N-methylglucamide; n-decyl β-D-glucopyranoside; n-decyl β-D-maltopyranoside; n-dodecyl β-D-glucopyranoside; n-dodecyl β-D-maltoside; heptanoyl-N-methylglucamide; n-heptyl-β-D-glucopyranoside; n-heptyl β-D-thioglucoside; n-hexyl β-D-glucopyranoside; nonanoyl-N-methylglucamide; n-noyl β-D-glucopyranoside; octanoyl-N-methylglucamide; n-octyl-β-D-glucopyranoside; octyl β-D-thioglucopyranoside; and the like. Most of these surfactants are known pharmaceutical excipients and are described in detail in the Handbook of Pharmaceutical Excipients, published jointly by the American Pharmaceutical Association and The Pharmaceutical Society of Great Britain (The Pharmaceutical Press, 1986), specifically incorporated by reference for such disclosure.

The hydrophobic, water-insoluble and water-indispersible polymer or copolymer may be chosen from biocompatible and biodegradable polymers, for example lactic or glycolic acid polymers and copolymers thereof, or polylactic/polyethylene (or polypropylene) oxide copolymers, preferably with molecular weights of between 1000 and 200000, polyhydroxybutyric acid polymers, polylactones of fatty acids containing at least 12 carbon atoms, or polyanhydrides.

The nanoparticles may be obtained by the technique of evaporation of solvent, from an aqueous dispersion or solution of phospholipids and of an oleic acid salt into which is added an immiscible organic phase comprising the active principle and the hydrophobic, water-insoluble and water-indispersible polymer or copolymer. The mixture is pre-emulsified and then subjected to homogenization and evaporation of the organic solvent to obtain an aqueous suspension of very small-sized nanoparticles.

A variety of methods are optionally employed to fabricate RNAi moleculesRNAi molecule nanoparticles that are within the scope of the embodiments. These methods include vaporization methods, such as free jet expansion, laser vaporization, spark erosion, electro explosion and chemical vapor deposition; physical methods involving mechanical attrition (e.g., “pearlmilling” technology, Elan Nanosystems), super critical CO₂ and interfacial deposition following solvent displacement. In one embodiment, the solvent displacement method is used. The size of nanoparticles produced by this method is sensitive to the concentration of polymer in the organic solvent; the rate of mixing; and to the surfactant employed in the process. Continuous flow mixers can provide the necessary turbulence to ensure small particle size. One type of continuous flow mixing device that is optionally used to prepare nanoparticles has been described (Hansen et al. J. Phys. Chem. 92, 2189-96, 1988). In other embodiments, ultrasonic devices, flow through homogenizers or supercritical CO2 devices may be used to prepare nanoparticles.

If suitable nanoparticle homogeneity is not obtained on direct synthesis, then size-exclusion chromatography is optionally used to produce highly uniform drug-containing particles that are freed of other components involved in their fabrication. Size-exclusion chromatography (SEC) techniques, such as gel-filtration chromatography, is optionally used to separate particle-bound RNAi molecules from non-particle bound RNAi molecules or to select a suitable size range of nanoparticles. Various SEC media, such as Superdex 200, Superose 6, Sephacryl 1000 are commercially available and are employed for the size-based fractionation of such mixtures. Additionally, nanoparticles is optionally purified by centrifugation, membrane filtration and by use of other molecular sieving devices, crosslinked gels/materials and membranes.

The present application also features a method for preparing dsRNA nanoparticles. In some embodiments, a first solution containing melamine derivatives is dissolved in an organic solvent such as dimethyl sulfoxide, or dimethyl formamide to which an acid such as HCl has been added. The concentration of HCl is about 3.3 moles of HCl for every mole of the melamine derivative. The first solution is then mixed with a second solution, which includes a nucleic acid dissolved or suspended in a polar or hydrophilic solvent (e.g., an aqueous buffer solution containing, for instance, ethylenediaminetraacetic acid (EDTA), or tris(hydroxymethyl)aminomethane (TRIS), or combinations thereof. The mixture forms a first emulsion. The mixing is done using any standard technique such as, for example sonication, vortexing, or in a microfluidizer. This causes complexing of the nucleic acids with the melamine derivative forming a trimeric nucleic acid complex. The concentration should be at least 1 to 7 moles of the melamine derivative for every mole of a double stranded nucleic acid having 20 nucleotide pairs, more if the ds nucleic acid is larger. The resultant nucleic acid particles are purified and the organic solvent removed (e.g., using size-exclusion chromatography or dialysis or both).

In some embodiments, the complexed nucleic acid nanoparticles are mixed with an aqueous solution containing either polyarginine, a Gln-Asn polymer, or both in an aqueous solution. This forms a solution containing nanoparticles of nucleic acid complexed with the melamine derivative and the polyarginine and/or the Gln-Asn polymers. In some embodiments, the molecular weight of polyarginine, and Gln-Asn polymers ranges from about 5000-15,000 Daltons. The mixing steps are carried out in a manner that minimizes shearing of the nucleic acid while producing nanoparticles on average smaller than 200 nanometers in diameter. In some embodiments, the polyarginine and/or the Gln-Asn polymer is present at a concentration of 2 moles per every mole of nucleic acid having 20 base pairs. In some embodiments, the concentration is increased proportionally for a nucleic acid having more than 20 base pairs.

In some embodiments, a nanoparticle disclosed herein is modified in order to direct binding of the nucleic acid complex to specific tissues. In some embodiments, an additional moiety (e.g., the TAT polypeptide, mannose or galactose) is covalently bound at either terminus of the polyarginine.

In some embodiments, a nanoparticle disclosed herein is purified by standard means such as size exclusion chromatography followed by dialysis.

In some embodiments, a nanoparticle disclosed herein is lyophilized using any suitable method.

Liposomes or lipid particles may also be employed to encapsulate the formulations or formulations. Phospholipids that are gently dispersed in an aqueous medium form multilayer vesicles with areas of entrapped aqueous media separating the lipid layers. Sonication, or turbulent agitation, of these multilayer vesicles results in the formation of single layer vesicles, commonly referred to as liposomes, with sizes of about 10-1000 nm. These liposomes have many advantages as carriers. They are biologically inert, biodegradable, non-toxic and non-antigenic. Liposomes are optionally formed in various sizes and with varying formulations and surface properties. Additionally, they are able to entrap a wide variety of agents and release the agent at the site of liposome collapse.

Suitable phospholipids for use in liposomes here are, for example, phosphatidyl cholines, ethanolamines and serines, sphingomyelins, cardiolipins, plasmalogens, phosphatictic acids and cerebrosides, in particular those which are soluble together with the RNAi molecules herein in non-toxic, pharmaceutically acceptable organic solvents. Preferred phospholipids are, for example, phosphatidyl choline, phosphatidyl ethanolmine, phosphatidyl serine, phosphatidyl inositol, lysophosphatidyl choline, phosphatidyl glycerol and the like, and mixtures thereof especially lecithin, e.g. soya lecithin. The amount of phospholipid used in the present formulation can range from about 10 to about 30%, preferably from about 15 to about 25% and in particular is about 20%.

Lipophilic additives may be employed advantageously to modify selectively the characteristics of the liposomes. Examples of such additives include by way of example only, stearylamine, phosphatictic acid, tocopherol, cholesterol, cholesterol hemisuccinate and lanolin extracts. The amount of lipophilic additive used can range from 0.5 to 8%, preferably from 1.5 to 4% and in particular is about 2%. Generally, the ratio of the amount of lipophilic additive to the amount of phospholipid ranges from about 1:8 to about 1:12 and in particular is about 1:10. Said phospholipid, lipophilic additive and the RNAi moleculesRNAi molecules are employed in conjunction with a non-toxic, pharmaceutically acceptable organic solvent system which can dissolve said ingredients. Said solvent system not only must dissolve the RNAi moleculesRNAi molecules completely, but it also has to allow the formulation of stable single bilayered liposomes. The solvent system comprises dimethylisosorbide and tetraglycol (glycofurol, tetrahydrofurfuryl alcohol polyethylene glycol ether) in an amount of about 8 to about 30%. In said solvent system, the ratio of the amount of dimethylisosorbide to the amount of tetraglycol can range from about 2:1 to about 1:3, in particular from about 1:1 to about 1:2.5 and preferably is about 1:2. The amount of tetraglycol in the final formulation thus can vary from 5 to 20%, in particular from 5 to 15% and preferably is approximately 10%. The amount of dimethylisosorbide in the final formulation thus can range from 3 to 10%, in particular from 3 to 7% and preferably is approximately 5%.

The term “organic component” as used hereinafter refers to mixtures comprising said phospholipid, lipophilic additives and organic solvents.

The RNAi moleculesRNAi molecules may be dissolved in the organic component, or other means to maintain full activity of the agent. The amount of RNAi molecules in the final formulation may range from 0.1 to 5.0%. In addition, other ingredients such as antioxidants may be added to the organic component. Examples include tocopherol, butylated hydroxyanisole, butylated hydroxytoluene, ascorbyl palmitate, ascorbyl oleate and the like.

Liposomal formulations are alternatively prepared, for RNAi molecules that are moderately heat-resistant, by (a) heating the phospholipid and the organic solvent system to about 60-80° C. in a vessel, dissolving the active ingredient, then adding any additional formulating agents, and stirring the mixture until complete dissolution is obtained; (b) heating the aqueous solution to 90-95° C. in a second vessel and dissolving the preservatives therein, allowing the mixture to cool and then adding the remainder of the auxiliary formulating agents and the remainder of the water, and stirring the mixture until complete dissolution is obtained; thus preparing the aqueous component; (c) transferring the organic phase directly into the aqueous component, while homogenizing the combination with a high performance mixing apparatus, in particular a high-shear mixer; and (d) adding a viscosity enhancing agent to the resulting mixture while further homogenizing. Preferably, the aqueous component is placed in a suitable vessel which is optionally equipped with a homogenizer and homogenization is effected by creating great turbulence during the injection of the organic component. Any mixing means or homogenizer which exerts high shear forces on the mixture may be employed. Generally, a mixer capable of speeds from about 1,500 to 20,000 rpm, in particular from about 3,000 to about 6,000 rpm may be employed. Suitable viscosity enhancing agents for use in process step (d) are for example, xanthan gum, hydroxypropyl cellulose, hydroxypropyl methylcellulose or mixtures thereof, cellulose derivatives being preferred. The amount of viscosity enhancing agent depends on the nature and the concentration of the other ingredients and in general ranges from about 0.5 to 1.5%, and in particular is approximately 1.5%. In order to prevent degradation of the materials used during the preparation of the liposomal formulation, it is advantageous to purge all solutions with an inert gas such as nitrogen or argon, and to conduct all steps under an inert atmosphere. Liposomes prepared by the above described method usually contain most of the active ingredient bound in the lipid bilayer and separation of the liposomes from unencapsulated material is not required.

### Dosage

The formulations comprising the RNAi molecules described herein are administered for prophylactic and/or therapeutic treatments. In therapeutic applications, the formulations are administered to a patient already suffering from a cancer in an amount sufficient to cure or at least partially arrest the symptoms of the cancer. Amounts effective for this use will depend on the severity and course of cancer, previous therapy, the patient's health status and response to the drugs, and the judgment of the treating physician.

In the case wherein the patient's condition does not improve, upon the doctor's discretion the administration of the RNAi molecule (e.g., siRNA molecules, miRNA molecules, and analogues thereof) may be administered chronically, that is, for an extended period of time, including throughout the duration of the patient's life in order to ameliorate or otherwise control or limit the symptoms of the patient's disease or condition.

In the case wherein the patient's status does improve, upon the doctor's discretion the administration of the RNAi molecule (e.g., siRNA molecules, miRNA molecules, and analogues thereof) may be given continuously; alternatively, the dose of drug being administered may be temporarily reduced or temporarily suspended for a certain length of time (i.e., a “drug holiday”). The length of the drug holiday can vary between 2 days and 1 year, including by way of example only, 2 days, 3 days, 4 days, 5 days, 6 days, 7 days, 10 days, 12 days, 15 days, 20 days, 28 days, 35 days, 50 days, 70 days, 100 days, 120 days, 150 days, 180 days, 200 days, 250 days, 280 days, 300 days, 320 days, 350 days, and 365 days. The dose reduction during a drug holiday may be from 10%-100%, including by way of example only 10%, 15%, 20%, 25%, 30%, 35%, 40%, 45%, 50%, 55%, 60%, 65%, 70%, 75%, 80%, 85%, 90%, 95%, and 100%.

Once improvement of the patient's conditions has occurred, a maintenance dose is administered if necessary. Subsequently, the dosage or the frequency of administration, or both, is optionally reduced, as a function of the symptoms, to a level at which the improved cancer is retained. In certain embodiments, patients require intermittent treatment on a long-term basis upon any recurrence of symptoms.

The amount of RNAi molecule (e.g., siRNA molecules, miRNA molecules, and analogues thereof) that will correspond to such an amount will vary depending upon factors such as the particular compound, cancer and its severity, according to the particular circumstances surrounding the case, including, e.g., the specific agent(s) being administered, the route of administration, the condition being treated, the target area being treated, and the subject or host being treated.

Typically, the dose range of the RNAi molecule (e.g., siRNA molecules, miRNA molecules, and analogues thereof) will be in the range of 0.001 to 500 milligrams per kilogram/day (e.g., about 1 microgram per kilogram to about 500 milligrams per kilogram, about 100 micrograms per kilogram to about 100 milligrams per kilogram, about 1 milligram per kilogram to about 75 milligrams per kilogram, about 10 micrograms per kilogram to about 50 milligrams per kilogram, or about 1 microgram per kilogram to about 50 micrograms per kilogram). Dosage levels of the order of from about 0.1 mg to about 140 mg per kilogram of body weight per day are useful in the treatment of the above-indicated conditions (about 0.5 mg to about 7 g per patient per day).

These and other effective unit dosage amounts may be administered in a single dose, or in the form of multiple daily, weekly or monthly doses, for example in a dosing regimen comprising from 1 to 5, or 2-3, doses administered per day, per week, or per month. The dosing schedule may vary depending on a number of clinical factors, such as the subject's sensitivity to the RNAi molecule. Examples of dosing schedules are 3 μg/kg administered twice a week, three times a week or daily; a dose of 7 μg/kg twice a week, three times a week or daily; a dose of 10 μg/kg twice a week, three times a week or daily; or a dose of 30 μg/kg twice a week, three times a week or daily.

The amount of the RNAi molecule (e.g., siRNA molecules, miRNA molecules, and analogues thereof) can vary, but in any event optimally will be an amount sufficient to target all atypical or malignant cells in the duct. Estimates of the quantity of target cells can be made upon the initial identification of the target duct (e.g. by cytological evaluation of ductal epithelial cells retrieved from the duct). The amount may vary depending on the agent's potency and other mitigating factors such as the extent of any time delay of delivery of the agent once inside the duct (e.g. with a time release formulation). Other factors such as whether the ductal epithelial cells are atypical or malignant (e.g. greater therapeutic activity may be needed for malignant cells), and/or how many genes might be affected by the methylation activity can also affect a determination of the amount of active agent to deliver to any given duct. The agent should be delivered in a sufficient amount to inhibit or reverse DNA methylation on promoters controlling genes transcribed and/or expressed in ductal epithelial cells of the target breast duct. Preferably the status of ductal markers and of the ductal epithelial cells will be evaluated prior to intraductal delivery of the demethylating and/or antimethylating agent(s), e.g. the evaluation can comprise MSP of the methylated genes (e.g to identify them and/or to quantify the amount of methylation) and/or cytological evaluation of the ductal epithelial cells (e.g. identify hyperplastic, atypical, or malignant cells).

### Combination Treatments

In certain embodiments, combinatorial formulations and coordinate administration methods are provided which employ an effective amount of an RNAi molecule, and a second therapeutic agent that is combinatorially formulated or coordinately administered with the RNAi molecule (e.g., siRNA molecules, miRNA molecules, and analogues thereof)—yielding an effective formulation or method to modulate, alleviate, treat or prevent the disease in a mammalian subject.

To practice the coordinate administration methods described herein, an RNAi molecule (e.g., siRNA molecules, miRNA molecules, and analogues thereof) is administered, simultaneously or sequentially, in a coordinate treatment protocol with a second therapeutic agent. The coordinate administration may be done in either order, and there may be a time period while only one or both (or all) active therapeutic agents, individually and/or collectively, exert their biological activities. A distinguishing aspect of all such coordinate treatment methods is that the RNAi molecule (e.g., siRNA molecules, miRNA molecules, and analogues thereof) present in the formulation elicits some favorable clinical response, which may or may not be in conjunction with a secondary clinical response provided by the secondary therapeutic agent. Often, the coordinate administration of the RNAi molecule (e.g., siRNA molecules, miRNA molecules, and analogues thereof) with a second therapeutic agent as contemplated herein will yield an enhanced therapeutic response beyond the therapeutic response elicited by either or both the purified RNAi molecule (e.g., siRNA molecules, miRNA molecules, and analogues thereof) and/or second therapeutic agent alone.

In some embodiments, the second agent is a demethylating agent (to remove existing hypermethylations), an inhibitor of DNA methylation (e.g. an agent comprising a moiety that competitively binds methyl groups and/or prevents methylation at cytosines) or an antagonist/inhibitor of DNA methyl transferase (the enzyme) or its activity leading to methylation of cytosines.

In some embodiments, the second therapeutic agent is selected from: cytotoxic agents, anti-angiogenesis agents and anti-neoplastic agents. In some embodiments, the second therapeutic agent is selected from alkylating agents, antimetabolites, epidophyllotoxins; antineoplastic enzymes, topoisomerase inhibitors, procarbazines, mitoxantrones, platinum coordination complexes, biological response modifiers and growth inhibitors, hormonal/anti-hormonal therapeutic agents, haematopoietic growth factors, aromatase inhibitors, anti-estrogens, anti-androgens, corticosteroids, gonadorelin agonists, microtubule active agents, nitrosoureas, lipid or protein kinase targeting agents, IMiDs, protein or lipid phosphatase targeting agents, anti-angiogenic agents, Akt inhibitors, IGF-I inhibitors, FGF3 modulators, mTOR inhibitors, Smac mimetics, HDAC inhibitors, agents that induce cell differentiation, bradykinin 1 receptor antagonists, angiotensin II antagonists, cyclooxygenase inhibitors, heparanase inhibitors, lymphokine inhibitors, cytokine inhibitors, IKK inhibitors, P38MAPK inhibitors, HSP90 inhibitors, multlikinase inhibitors, bisphosphanate, rapamycin derivatives, anti-apoptotic pathway inhibitors, apoptotic pathway agonists, PPAR agonists, RAR agonists, inhibitors of Ras isoforms, telomerase inhibitors, protease inhibitors, metalloproteinase inhibitors, aminopeptidase inhibitors, SHIP activators—AQX-MN100, Humax-CD20 (ofatumumab), CD20 antagonists, IL2-diptheria toxin fusions, or combinations thereof.

In some embodiments, the second therapeutic agent is selected from ARRY-797, dacarbazine (DTIC), actinomycins C₂, C₃, D, and F₁, cyclophosphamide, melphalan, estramustine, maytansinol, rifamycin, streptovaricin, doxorubicin, daunorubicin, epirubicin, idarubicin, detorubicin, carminomycin, idarubicin, epirubicin, esorubicin, mitoxantrone, bleomycins A, A₂, and B, camptothecin, Irinotecan, Topotecan, 9-aminocamptothecin, 10,11-methylenedioxycamptothecin, 9-nitrocamptothecin, bortezomib, temozolomide, TAS 103, NPI0052, combretastatin, combretastatin A-2, combretastatin A-4, calicheamicins, neocarcinostatins, epothilones A B, C, and semi-synthetic variants, Herceptin, Rituxan, CD40 antibodies, asparaginase, interleukins, interferons, leuprolide, and pegaspargase, 5-fluorouracil, fluorodeoxyuridine, ptorafur, 5′-deoxyfluorouridine, UFT, MITC, S-1 capecitabine, diethylstilbestrol, tamoxifen, toremefine, tolmudex, thymitaq, flutamide, fluoxymesterone, bicalutamide, finasteride, estradiol, trioxifene, dexamethasone, leuproelin acetate, estramustine, droloxifene, medroxyprogesterone, megesterol acetate, aminoglutethimide, testolactone, testosterone, diethylstilbestrol, hydroxyprogesterone, mitomycins A, B and C, porfiromycin, cisplatin, carboplatin, oxaliplatin, tetraplatin, platinum-DACH, ormaplatin, thalidomide, lenalidomide, CI-973, telomestatin, CHIR258, Rad 001, SAHA, Tubacin, 17-AAG, sorafenib, JM-216, podophyllotoxin, epipodophyllotoxin, etoposide, teniposide, Tarceva, Iressa, Imatinib, Miltefosine, Perifosine, aminopterin, methotrexate, methopterin, dichloro-methotrexate, 6-mercaptopurine, thioguanine, azattuoprine, allopurinol, cladribine, fludarabine, pentostatin, 2-chloroadenosine, deoxycytidine, cytosine arabinoside, cytarabine, azacitidine, 5-azacytosine, gencitabine, 5-azacytosine-arabinoside, vincristine, vinblastine, vinorelbine, leurosine, leurosidine and vindesine, paclitaxel, taxotere and docetaxel.

In some embodiments, the second therapeutic agent is selected from corticosteroids, non-steroidal anti-inflammatories, muscle relaxants and combinations thereof with other agents, anaesthetics and combinations thereof with other agents, expectorants and combinations thereof with other agents, antidepressants, anticonvulsants and combinations thereof; antihypertensives, opioids, topical cannabinoids, capsaicin, betamethasone dipropionate (augmented and nonaugmented), betamethasone valerate, clobetasol propionate, prednisone, methyl prednisolone, diflorasone diacetate, halobetasol propionate, amcinonide, dexamethasone, dexosimethasone, fluocinolone acetononide, fluocinonide, halocinonide, clocortalone pivalate, dexosimetasone, flurandrenalide, salicylates, ibuprofen, ketoprofen, etodolac, diclofenac, meclofenamate sodium, naproxen, piroxicam, celecoxib, cyclobenzaprine, baclofen, cyclobenzaprine/lidocaine, baclofen/cyclobenzaprine, cyclobenzaprine/lidocaine/ketoprofen, lidocaine, lidocaine/deoxy-D-glucose, prilocaine, EMLA Cream (Eutectic Mixture of Local Anesthetics (lidocaine 2.5% and prilocaine 2.5%), guaifenesin, guaifenesin/ketoprofen/cyclobenzaprine, amitryptiline, doxepin, desipramine, imipramine, amoxapine, clomipramine, nortriptyline, protriptyline, duloxetine, mirtazepine, nisoxetine, maprotiline, reboxetine, fluoxetine, fluvoxamine, carbamazepine, felbamate, lamotrigine, topiramate, tiagabine, oxcarbazepine, carbamezipine, zonisamide, mexiletine, gabapentin/clonidine, gabapentin/carbamazepine, carbamazepine/cyclobenzaprine, antihypertensives including clonidine, codeine, loperamide, tramadol, morphine, fentanyl, oxycodone, hydrocodone, levorphanol, butorphanol, menthol, oil of wintergreen, camphor, eucalyptus oil, turpentine oil; CB1/CB2 ligands, acetaminophen, infliximab, nitric oxide synthase inhibitors, particularly inhibitors of inducible nitric oxide synthase, PDE4 inhibitors—similar mechanism to Ibudilast (AV-411), CDC-801, JNK inhibitors—CC-401, Combination TNF/PDE4 inhibitors—CDC-998, IL1 antagonists e.g. Anakinra—Kineret, AMG 108, (mAb) that targets IL-1, SHIP activators—AQX-MN100, C5 antagonists, C5a inhibitors, Pexelizumab, Pyrimidine synthesis inhibitors, lymphokine inhibitors, cytokine inhibitors, IKK inhibitors, P38MAPK inhibitors, ARRY-797, HSP90 inhibitors, multlikinase inhibitors, bisphosphanates, PPAR agonists, Cox1 and cox 2 inhibitors, Anti-CD4 therapy, B-cell inhibitors, COX/LOX dual inhibitors, Immunosuppressive agents, iNOS inhibitors, NSAIDs, sPLA2 inhibitors, Colchicine, allopurinol, oxypurinol, Gold, Ridaura—Auranofin, febuxostat, Puricase, PEG-uricase formulations, Benzbromarone, Long-acting beta-2 agonists (LABA5), salmeterol (Serevent Diskus) and formoterol (Foradil), Leukotriene modifiers include montelukast (Singulair) and zafirlukast (Accolate). Inhaled cromolyn (Intal) or nedocromil (Tilade), Theophylline. Short-acting beta-2 agonists, Ipratropium (Atrovent), Immunotherapy-(Allergy-desensitization shots), Anti-IgE monoclonal antibodies—Xolair, Common DMARDs include hydroxychloroquine (Plaquenil), the gold compound auranofin (Ridaura), sulfasalazine (Azulfidine), minocycline (Dynacin, Minocin) and methotrexate (Rheumatrex), leflunomide (Arava), azathioprine (Imuran), cyclosporine (Neoral, Sandimmune) and cyclophosphamide (Cytoxan), Antibiotics, CD80 antagonists, costimulatory factor antagonists, Humax-CD20 (ofatumumab); CD20 antagonists, MEK inhibitors, NF kappa B inhibitors, anti B-cell antibodies, denosumab, mAb that specifically targets the receptor activator of nuclear factor kappa B ligand (RANKL). IL17 inactivating anti-bodies, IL-17 receptor antagonists/inhibitors, CTLA inhibitors, CD20 inhibitors, soluble VEGFR-1 receptors, anti-VEGFR-1 receptor antibodies, anti-VEGF antibodies, integrin receptor antagonist, Selectin inhibitors, P-selectin and E-selectin inhibitors, Phospholipase A2 Inhibitors, Lipoxygenase Inhibitors, RANKL and RANK antagonists/antibodies, Osteoprotegerin antagonists, Lymphotoxin inhibitors, B-lymphocyte stimulator, MCP-1 inhibitors, MIF inhibitors, inhibitors of: CD2, CD3, CD4, CD25, CD40 and CD40 Ligand CD152 (CTLA4), Macrolide immunosuppressants, Selective inhibitors of nucleotide metabolism, Inhibitors of chemotaxis, CXC receptor and CXC ligand inhibitors, Chemokine Antagonists, leukocyte chemotaxis inhibitors Adhesion Molecule blockers, Selectins Lymphocyte Function Antigen-1 (LFA-1, CD11a) antagonists, Very Late Antigen-4 (VLA-4) antagonists, Matrix Metalloprotease Inhibitors, Elastase Inhibitors, Cathepsin Inhibitors.

In some embodiments, the second therapeutic agent is selected from beta-blockers, carbonic anhydrase inhibitors, α- and β-adrenergic antagonists including al-adrenergic antagonists, α2 agonists, miotics, prostaglandin analogs, corticosteroids, and immunosuppressant agents. In some embodiments, the second therapeutic agent is selected from timolol, betaxolol, levobetaxolol, carteolol, levobunolol, propranolol, brinzolamide, dorzolamide, nipradilol, iopidine, brimonidine, pilocarpine, epinephrine, latanoprost, travoprost, bimatoprost, unoprostone, dexamethasone, prednisone, methylprednisolone, azathioprine, cyclosporine, and immunoglobulins.

In some embodiments, the second therapeutic agent is selected from corticosteroids, immunosuppressants, prostaglandin analogs and antimetabolites. In some embodiments, the second therapeutic agent is selected from dexamethasome, prednisone, methylprednisolone, azathioprine, cyclosporine, immunoglobulins, latanoprost, travoprost, bimatoprost, unoprostone, infliximab, rutuximab, methotrexate, non-steroidal anti-inflammatories, muscle relaxants and combinations thereof with other agents, anaesthetics and combinations thereof with other agents, expectorants and combinations thereof with other agents, antidepressants, anticonvulsants and combinations thereof; antihypertensives, opioids, topical cannabinoids, and other agents, such as capsaicin, betamethasone dipropionate (augmented and nonaugmented), betamethasone valerate, clobetasol propionate, prednisone, methyl prednisolone, diflorasone diacetate, halobetasol propionate, amcinonide, dexamethasone, dexosimethasone, fluocinolone acetononide, fluocinonide, halocinonide, clocortalone pivalate, dexosimetasone, flurandrenalide, salicylates, ibuprofen, ketoprofen, etodolac, diclofenac, meclofenamate sodium, naproxen, piroxicam, celecoxib, cyclobenzaprine, baclofen, cyclobenzaprine/lidocaine, baclofen/cyclobenzaprine, cyclobenzaprine/lidocaine/ketoprofen, lidocaine, lidocaine/deoxy-D-glucose, prilocaine, EMLA Cream (Eutectic Mixture of Local Anesthetics (lidocaine 2.5% and prilocaine 2.5%), guaifenesin, guaifenesin/ketoprofen/cyclobenzaprine, amitryptiline, doxepin, desipramine, imipramine, amoxapine, clomipramine, nortriptyline, protriptyline, duloxetine, mirtazepine, nisoxetine, maprotiline, reboxetine, fluoxetine, fluvoxamine, carbamazepine, felbamate, lamotrigine, topiramate, tiagabine, oxcarbazepine, carbamezipine, zonisamide, mexiletine, gabapentin/clonidine, gabapentin/carbamazepine, carbamazepine/cyclobenzaprine, antihypertensives including clonidine, codeine, loperamide, tramadol, morphine, fentanyl, oxycodone, hydrocodone, levorphanol, butorphanol, menthol, oil of wintergreen, camphor, eucalyptus oil, turpentine oil; CB1/CB2 ligands, acetaminophen, infliximab; nitric oxide synthase inhibitors, particularly inhibitors of inducible nitric oxide synthase; and other agents, such as capsaicin. PDE4 inhibitors—similar mechanism to Ibudilast (AV-411), CDC-801, JNK inhibitors—CC-401, Combination TNF/PDE4 inhibitors—CDC-998, IL1 antagonists e.g. Anakinra—Kineret, AMG 108, (mAb) that targets IL-1, SHIP activators—AQX-MN100, C5 antagonists, C5a inhibitors, Pexelizumab, Pyrimidine synthesis inhibitors, lymphokine inhibitors, cytokine inhibitors, IKK inhibitors, P38MAPK inhibitors, ARRY-797, HSP90 inhibitors, multlikinase inhibitors, bisphosphanates, PPAR agonists, Cox1 and cox 2 inhibitors, Anti-CD4 therapy, B-cell inhibitors, COX/LOX dual inhibitors, Immunosuppressive agents, iNOS inhibitors, NSAIDs, sPLA2 inhibitors, Colchicine, allopurinol, oxypurinol, Gold, Ridaura—Auranofin, febuxostat, Puricase, PEG-uricase formulations, Benzbromarone, Long-acting beta-2 agonists (LABA5), salmeterol (Serevent Diskus) and formoterol (Foradil), Leukotriene modifiers include montelukast (Singulair) and zafirlukast (Accolate). Inhaled cromolyn (Intal) or nedocromil (Tilade), Theophylline. Short-acting beta-2 agonists, Ipratropium (Atrovent), Immunotherapy-(Allergy-desensitization shots), Anti-IgE monoclonal antibodies—Xolair, Common DMARDs include hydroxychloroquine (Plaquenil), the gold compound auranofin (Ridaura), sulfasalazine (Azulfidine), minocycline (Dynacin, Minocin) and methotrexate (Rheumatrex), leflunomide (Arava), azathioprine (Imuran), cyclosporine (Neoral, Sandimmune) and cyclophosphamide (Cytoxan), Antibiotics, CD80 antagonists, costimulatory factor antagonists, Humax-CD20 (ofatumumab); CD20 antagonists, MEK inhibitors, NF kappa B inhibitors, anti B-cell antibodies, denosumab, mAb that specifically targets the receptor activator of nuclear factor kappa B ligand (RANKL). IL17 inactivating anti-bodies, IL-17 receptor antagonists/inhibitors, CTLA inhibitors, CD20 inhibitors, soluble VEGFR-1 receptors, anti-VEGER-1 receptor antibodies, anti-VEGF antibodies, integrin receptor antagonist, Selectin inhibitors, P-selectin and E-selectin inhibitors, Phospholipase A2 Inhibitors, Lipoxygenase Inhibitors, RANKL and RANK antagonists/antibodies, Osteoprotegerin antagonists, Lymphotoxin inhibitors, B-lymphocyte stimulator, MCP-1 inhibitors, MIF inhibitors, inhibitors of: CD2, CD3, CD4, CD25, CD40 and CD40 Ligand CD152 (CTLA4), Macrolide immunosuppressants, Selective inhibitors of nucleotide metabolism, Inhibitors of chemotaxis, CXC receptor and CXC ligand inhibitors, Chemokine Antagonists, leukocyte chemotaxis inhibitors Adhesion Molecule blockers, Selectins Lymphocyte Function Antigen-1 (LFA-1, CD11a) antagonists, Very Late Antigen-4 (VLA-4) antagonists, Matrix Metalloprotease Inhibitors, Elastase Inhibitors, Cathepsin Inhibitors.

In some embodiments, the second therapeutic agent is selected from insulin, insulin derivatives and mimetics, insulin secretagogues, insulin sensitizers, biguanide agents, alpha-glucosidase inhibitors, insulinotropic sulfonylurea receptor ligands, protein tyrosine phosphatase-1B (PTP-1B) inhibitors, GSK3 (glycogen synthase kinase-3) inhibitors, GLP-1 (glucagon like peptide-1), GLP-1 analogs, DPPIV (dipeptidyl peptidase IV) inhibitors, RXR ligands sodium-dependent glucose co-transporter inhibitors, glycogen phosphorylase A inhibitors, an AGE breaker, PPAR modulators, LXR and FXR modulators, non-glitazone type PPARS agonist, selective glucocorticoid antagonists, metformin, Glipizide, glyburide, Amaryl, meglitinides, nateglinide, repaglinide, PT-112, SB-517955, SB4195052, SB-216763, NN-57-05441, NN-57-05445, GW-0791, AGN-.sup.194.sup.204, T-1095, BAY R3401, acarbose Exendin-4, DPP728, LAF237, vildagliptin, MK-0431, saxagliptin, GSK23A, pioglitazone, rosiglitazone, (R)-1-{4-[5-methyl-2-(4-trifluoromethyl-phenyl)-oxazol-4-ylmethoxy]-benze-nesulfonyl}2,3-dihydro-1H-indole-2-carboxylic acid described in the patent application WO 03/043985, as compound 19 of Example 4, and GI-262570.

While a number of embodiments have been shown and described herein, it will be apparent that such embodiments are provided by way of example only. Numerous variations, changes, and substitutions will now occur to those skilled in the art without departing from the described embodiments. It should be understood that various alternatives to the embodiments described herein may be employed. It is intended that the following claims and their equivalents be covered thereby.

### EXAMPLES

### Example 1

### Breast Cancer

A breast duct on the right breast of a patient is identified as having malignancy tumor. Four genes are tested in ductal epithelial cells retrieved from the tumor by methylation specific PCR (MSP) to further establish a methylated state of some promoters of some genes transcribed and/or expressed in the ductal environment. It is found that RARβ2, twist, maspin, and cyclin D2 are all expressed in the ductal epithelium that show some percentage of methylation on the promoter CpG islands as indicated by MSP.

A formulation comprising a siRNA molecule with several glycerol nucleic acid substitutions targeting the CpG regions on the various promoters of the various target genes and a Krebs Cycle analogue carrier is administered directly into the breast duct tumor once a week for 1 month.

The breast duct is analyzed one month following administration of the formulation. Tumor size is determined.

### Example 2

### Colon Cancer

A patient with colon cancer is identified.

A formulation comprising a siRNA molecule with several glycerol nucleic acid substitutions targeting the MSH2 gene and a Krebs Cycle analogue carrier is administered intravenously once a week for 1 month.

The colon is analyzed one month following administration of the formulation. Tumor size is determined.

### Example 3

### Lung Cancer

A patient with lung cancer is identified.

A formulation comprising a siRNA molecule with several glycerol nucleic acid substitutions targeting the PI3K gene and a Krebs Cycle analogue carrier is administered intravenously once a week for 1 month.

The lung is analyzed one month following administration of the formulation. Tumor size is determined

### Example 4

### Prostate Cancer

A patient with prostate cancer is identified.

A formulation comprising a siRNA molecule with several glycerol nucleic acid substitutions targeting the PCA3gene and a Krebs Cycle analogue carrier is administered intravenously once a week for 1 month.

The lung is analyzed one month following administration of the formulation. Tumor size is determined

### Example 5

### Combination Treatment for Breast Cancer

A patient with breast cancer is identified. Tumor size is measured.

A formulation comprising (a) an RNAi molecule targeting the CpG region on the promoters of RARβ2, and (b) a Krebs Cycle analogue carrier is administered once every two weeks for 2 months. After administration of the RNAi molecule, tamoxifen is administered.

Tumor size is analyzed at the end of the two months.

### Example 6

### Formulation

An RNAi molecule targeting the CpG region on the promoters of RARβ2 is synthesized. The molecule contains several glycerol nucleic acid substitutions.

The RNAi molecule is mixed with a Krebs Cycle analogue carrier.

The RNAi molecule/carrier solution is diluted in Ringer's solution.

### Example 7

### Nanoparticle Formulation

A first solution containing melamine derivatives is dissolved in dimethyl sulfoxide, to which HCl has been added. The concentration of HCl is about 3.3 moles of HCl for every mole of the melamine derivative.

A second solution containing an RNAi molecule targeting BRCA1 dissolved in ethylenediaminetraacetic acid (EDTA) is prepared.

The first solution is then mixed with a second solution. The mixture forms a first emulsion. The mixing is done via sonication. The RNAi molecule complexes with the dimethyl sulfoxide forming a trimeric nucleic acid complex.

The resultant nucleic acid particles are purified using size-exclusion chromatography

What is claimed is:

1. A formulation, comprising:
(a) an RNAi molecule comprising at least on: locked nucleic acid (LNA), unlocked nucleic acid (UNA), bridged nucleic acid (BNA), glycerol nucleic acid (GNA), or a combination thereof; and
(b) an RNAi carrier.

2. The formulation of claim 1, wherein the carrier provides for one or more of the following: stability for shortened duplexes, reduction or prevention of sense strand loading, reduction or prevention of seed region microRNA adverse side effects and reduction of non-specific immunoactivation.

3. The formulation of claim 1, wherein the RNAi carrier is a di-lipid amino acid (DILA²).

4. The formulation of claim 1, wherein the RNAi carrier is a Krebs Cycle analog.

5. The formulation of claim 1, wherein the RNAi carrier is a Krebs Cycle analog and wherein the Krebs Cycle analog reduces or prevents cytotoxicity.

6. A formulation, comprising:
(a) an RNAi molecule; and
(b) a Krebs Cycle analog RNAi carrier.

7. The formulation of claim 6, wherein the RNA or RNA analog comprises a locked nucleic acid (LNA), an unlocked nucleic acid (UNA), a bridged nucleic acid (BNA), a glycerol nucleic acid (GNA), or a combination thereof.

8. A formulation, comprising:
(a) an RNAi molecule comprising at least on: locked nucleic acid (LNA), unlocked nucleic acid (UNA), bridged nucleic acid (BNA), glycerol nucleic acid (GNA), or a combination thereof; and
(b) a Krebs Cycle analog RNAi carrier.

9. A formulation, comprising:
(a) an RNAi molecule comprising at least on glycerol nucleic acid (GNA), or a combination thereof a glycerol nucleic acid (GNA) analog; and
(b) a Krebs Cycle analog RNAi carrier.

10. Use of a formulation of any of claims 1-9 for the manufacture of a medicament for the treatment of cancer.

11. Use of a formulation of any of claims 1-9 for the manufacture of a medicament for inducing apoptosis of a circulating tumor cell (CTC).

12. Use of a formulation of any of claims 1-9 for the treatment of cancer.

13. The use of claim 12, wherein the cancer is characterized by the presence of a primary tumor or a metastasis.

14. The use of claim 12, wherein the cancer is breast cancer, a gastrointestinal cancer (such as a colon cancer), lung cancer or prostate cancer.

15. The use of claim 12, wherein the formulation is administered before, during, or immediately after surgery to remove a primary tumor or a metastasis.

16. The use of claim 15, wherein the formulation is locally administered at the site of the surgery.

17. The use of claim 12, wherein the formulation is administered in a time-release formulation.

18. The use of claim 12, wherein the formulation is administered by intravenous injection.

19. The use of claim 12, wherein the formulation exhibits reduced lipid-induced hepatic toxicity.

20. The use of claim 12, wherein the formulation reduces spread of the primary tumor or metastases.

21. Use of a formulation of any of claims 1-9 for inducing apoptosis of a circulating tumor cell (CTC).

22. The use of claim 21, wherein the circulating tumor cell (CTC) is from a primary tumor or a metastasis.

23. The use of claim 21, wherein the formulation is administered in a time-release formulation.

24. The use of claim 21, wherein the formulation is administered by intravenous injection.

25. The use of claim 21, wherein the formulation is administered in a time-release formulation and by intravenous injection.

26. The use of claim 21, wherein the formulation exhibits reduced lipid-induced hepatic toxicity.

27. Use of a formulation of any of claims 1-9 for inhibiting cancerous and pre-cancerous gene expression of breast cancer-related genes and pre-cancerous-related genes.

28. The use of claim 27, wherein the formulation is administered to an individual presenting with premalignant or malignant breast duct epithelial cells in a breast duct.

29. The use of claim 27, wherein the formulation is administered locally the breast duct.

30. The use of claim 27, wherein the formulation is administered in a time-release formulation.
